(19) United States
(12) Patent Application Publication
(10) Pub. No.: US 2014/0012146 A1
(43) Pub. Date: Jan. 9, 2014

(54) MEASUREMENT APPARATUS, MEASUREMENT METHOD, PROGRAM, STORAGE MEDIUM, AND MEASUREMENT SYSTEM

(71) Applicant: Sony Corporation, Tokyo (JP)

(72) Inventor: Kunio Fukuda, Tokyo (JP)

(21) Appl. No.: 13/926,342

(22) Filed: Jun. 25, 2013

(30) Foreign Application Priority Data
Jul. 4, 2012 (JP) 2012-150244

(51) Int. Cl. A61B 5/021; A61B 5/0402

(52) U.S. Cl. CPC A61B 5/02108; A61B 5/0402
USPC 600/485

(57) ABSTRACT

There is provided a measurement apparatus including a blood pressure calculation unit configured to calculate a blood pressure value based on electrocardiography information relating to an electrocardiogram of a measurement subject and pulse wave information relating to a pulse wave of the measurement subject, and a chest contact measurement unit that includes an electrocardiography measurement unit that is brought into contact with a chest of the measurement subject to measure the electrocardiogram and a pulse wave measurement unit configured to measure the pulse wave from a pulse wave detection site of the measurement subject.

### BACKGROUND

The present disclosure relates to a measurement apparatus, a measurement method, a program, a storage medium, and a measurement system.

Conventionally, as a method for measuring blood pressure, a direct measurement method has been known in which blood pressure is directly measured by utilizing air pressure. In this direct measurement method, pressure is applied on the blood vessels by supplying air through an air pump into a tube, called a cuff, that is wrapped around an arm or the like. By adjusting the air flow supplied into the cuff to change the pressure applied on the blood vessels, the pressure value when the blood starts or stop flowing is determined, whereby blood pressure is measured. However, since a direct measurement system blood pressure monitor has to have a cuff, an air pump, a detection device for detecting the start and stop of blood flowing and the like, such a monitor is not suited to portable applications. Further, since a direct measurement system blood pressure monitor takes effort and time for measurement, measuring blood pressure casually on a daily basis is difficult.

Accordingly, a so-called pulse wave system blood pressure monitor has been proposed that utilizes pulse wave velocity to measure blood pressure. For example, JP-A-2002-172094 discloses a wristwatch type blood pressure monitor that measures blood pressure by calculating pulse wave velocity that is based on an electrocardiography waveform (electrocardiogram) measured by bringing the arm and finger into contact with an electrode, and a pulse wave measured at the finger. Further, JP-A-2004-201868 discloses a wristwatch type blood pressure monitor that measures blood pressure based on a time difference between a pulse wave measured at the blood vessels in the arm and a pulse wave measured at the blood vessels in the finger.

### SUMMARY

However, since there are individual differences in the position and angle of a person's heart, with the technology disclosed in JP-A-2002-172094, depending on the measurement subject, in some cases the electrocardiography waveform cannot be measured. Further, since the blood pressure monitor disclosed in JP-A-2002-172094 is a wristwatch type, if there is loosening of the belt or the like, the contact between the electrodes and the measurement site is insufficient, which can prevent measurement from being performed accurately.

Further, with the technology disclosed in JP-A-2004-201868, since a cable is provided for transmitting pulse wave-related data measured at a finger to the blood pressure monitor main body that is worn on the user's arm, from the perspective of user friendliness for the subject, it is difficult to continuously wear on a daily basis. Moreover, since the blood pressure monitor disclosed in JP-A-2004-201868 is a wristwatch type, similar to the blood pressure monitor disclosed in JP-A-2002-172094, if there is loosening of the belt or the like, measurement may not be performed accurately.

According to an embodiment of the present disclosure, there is provided a novel and improved measurement apparatus, measurement method, program, storage medium, and measurement system that are capable of realizing a more accurate blood pressure measurement and better user convenience.

According to an embodiment of the present disclosure, there is provided a measurement apparatus including a blood pressure calculation unit configured to calculate a blood pressure value based on electrocardiography information relating to an electrocardiogram of a measurement subject and pulse wave information relating to a pulse wave of the measurement subject, and a chest contact measurement unit that includes an electrocardiography measurement unit that is brought into contact with a chest of the measurement subject to measure the electrocardiogram and a pulse wave measurement unit configured to measure the pulse wave from a pulse wave detection site of the measurement subject.

According to an embodiment of the present disclosure, there is provided a measurement method including acquiring pulse wave information relating to a pulse wave of a measurement subject and electrocardiography information relating to an electrocardiogram of the measurement subject input from an electrocardiography measurement unit in contact with a chest of the measurement subject, and calculating a blood pressure value based on the pulse wave information and the electrocardiography information.

According to an embodiment of the present disclosure, there is provided a program that causes a computer to realize a blood pressure calculation function of calculating a blood pressure value based on electrocardiography information relating to an electrocardiogram of a measurement subject and pulse wave information relating to a pulse wave of the measurement subject, and a chest contact measurement function that includes an electrocardiography measurement unit that is brought into contact with a chest of the measurement subject to measure the electrocardiogram and a pulse wave measurement unit configured to measure the pulse wave from a pulse wave detection site of the measurement subject.

According to an embodiment of the present disclosure, there is provided a computer-readable recording medium on which is recorded a program that causes a computer to realize a blood pressure calculation function of calculating a blood pressure value based on electrocardiography information relating to an electrocardiogram of a measurement subject and pulse wave information relating to a pulse wave of the measurement subject, and a chest contact measurement function that includes an electrocardiography measurement unit that is brought into contact with a chest of the measurement subject to measure the electrocardiogram and a pulse wave measurement unit configured to measure the pulse wave from a pulse wave detection site of the measurement subject.

According to an embodiment of the present disclosure, there is provided a measurement system including a blood pressure calculation unit configured to calculate a blood pressure value based on electrocardiography information relating to an electrocardiogram of a measurement subject and pulse wave information relating to a pulse wave of the measurement subject, and a chest contact measurement unit that includes an electrocardiography measurement unit that is brought into contact with a chest of the measurement subject to measure the electrocardiogram and a pulse wave measurement unit configured to measure the pulse wave from a pulse wave detection site of the measurement subject.

According to an embodiment of the present disclosure, there is provided a measurement system including a calculation server that includes a blood pressure calculation unit configured to calculate a blood pressure value based on electrocardiography information relating to an electrocardiogram of a measurement subject and pulse wave information relating to a pulse wave of the measurement subject, and a measurement apparatus including a chest contact measurement unit that includes an electrocardiography measurement unit that is brought into contact with a chest of the measurement subject to measure the electrocardiogram and a pulse wave measurement unit configured to measure the pulse wave from a pulse wave detection site of the measurement subject.

According to the embodiments of the present disclosure described above, a chest contact measurement unit includes an electrocardiography measurement unit and a pulse wave measurement unit. The electrocardiography measurement unit measures an electrocardiogram when brought into contact with the chest of the measurement subject, and the pulse wave measurement unit measures a pulse wave from a pulse wave detection site of the measurement subject. Further, a blood pressure calculation unit calculates a blood pressure value based on electrocardiography information relating to the electrocardiogram of the measurement subject and pulse wave information relating to the pulse wave of the measurement subject.

Thus, according to the embodiments of the present disclosure described above, a more accurate blood pressure measurement and better user convenience can be realized.

### BRIEF DESCRIPTION OF THE DRAWINGS

FIG. 1A is a schematic diagram illustrating a configuration example of a conventional pulse wave system blood pressure monitor;

FIG. 1B is a schematic diagram illustrating a configuration example of a conventional pulse wave system blood pressure monitor;

FIG. 1C is a schematic diagram illustrating a configuration example of a conventional pulse wave system blood pressure monitor;

FIG. 2 is a function block diagram illustrating a schematic configuration of a measurement apparatus according to a first embodiment of the present disclosure;

FIG. 3 is a function block diagram illustrating a schematic configuration of a chest contact measurement unit of the measurement apparatus illustrated in FIG. 2;

FIG. 4 is a graph that plots an electrocardiography waveform and a pulse wave;

FIG. 5 is an expanded diagram illustrating a period close to time T1 and time T2 in FIG. 4 in an expanded manner;

FIG. 6 is a diagram illustrating a relationship between pulse wave velocity and a systolic pressure (maximum blood pressure) value;

FIG. 7 is a schematic diagram illustrating an example of a blood pressure monitor for calibration;

FIG. 8A is a rear view illustrating an appearance example of a measurement apparatus according to an embodiment of the present disclosure;

FIG. 8B is a front view illustrating an appearance example of a measurement apparatus according to an embodiment of the present disclosure;

FIG. 8C is a bottom view illustrating an appearance example of a measurement apparatus according to an embodiment of the present disclosure;

FIG. 8D is a side view illustrating an appearance example of a measurement apparatus according to an embodiment of the present disclosure;

FIG. 8E is an explanatory diagram illustrating a positional relationship between the measurement apparatus according to an embodiment of the present disclosure and a pulse wave measurement site when measuring a pulse wave;

FIG. 9A is a rear view illustrating an appearance example of a dry electrode according to an embodiment of the present disclosure;

FIG. 9B is a top view of the dry electrode illustrated in FIG. 9A;

FIG. 9C is a rear view illustrating an appearance example of a wet electrode according to an embodiment of the present disclosure;

FIG. 9D is a top view of the wet electrode illustrated in FIG. 9C;

FIG. 10A is an explanatory diagram illustrating an example of a usage method when the measurement apparatus according to an embodiment of the present disclosure has a dry electrode;

FIG. 10B is an explanatory diagram illustrating an example of a usage method when the measurement apparatus according to an embodiment of the present disclosure has a wet electrode;

FIG. 11A is a rear view illustrating an appearance example of a measurement apparatus according to a second embodiment of the present disclosure;

FIG. 11B is a front view illustrating an appearance example of a measurement apparatus according to a second embodiment of the present disclosure;

FIG. 11C is a side view illustrating an appearance example of a measurement apparatus according to a second embodiment of the present disclosure;

FIG. 11D is an explanatory diagram illustrating a positional relationship between the measurement apparatus according to a second embodiment of the present disclosure and a pulse wave measurement site when measuring a pulse wave;

FIG. 12 is an explanatory diagram illustrating an example of a usage method when the measurement apparatus according to a second embodiment of the present disclosure has a dry electrode;

FIG. 13 is a flow diagram illustrating a processing procedure of a measurement method according to a first and a second embodiment of the present disclosure;

FIG. 14 is a function block diagram illustrating an example of a different configuration of a measurement apparatus according to a first and a second embodiment of the present disclosure;

FIG. 15 is a function block diagram illustrating a schematic configuration of a chest contact measurement unit of the measurement apparatus illustrated in FIG. 14;

FIG. 16 is a rear view illustrating an appearance example of the measurement apparatus illustrated in FIG. 14;

FIG. 17 is a function block diagram illustrating a schematic configuration of a measurement system according to a first and a second embodiment of the present disclosure;

FIG. 18A is a schematic diagram illustrating how the measurement apparatus of a measurement system according to a first and a second embodiment of the present disclosure looks when held in both hands by a measurement subject;

FIG. 18B is an expanded view illustrating how the hands of the measurement subject in FIG. 18A look from the measurement subject side;

FIG. 18C is an expanded view illustrating how the hands of the measurement subject in FIG. 18A look from the reverse side of the measurement subject; and

FIG. 19 is a function block diagram illustrating an example of a hardware configuration of an information processing apparatus according to a first and a second embodiment of the present disclosure.

### DETAILED DESCRIPTION OF THE EMBODIMENT(S)

Hereinafter, preferred embodiments of the present disclosure will be described in detail with reference to the appended drawings. Note that, in this specification and the appended drawings, structural elements that have substantially the same function and structure are denoted with the same reference numerals, and repeated explanation of these structural elements is omitted.

The description will now be made in the following order.

1. Investigations by the inventor
2. First embodiment of the present disclosure
2.1. Configuration of the measurement apparatus
2.2. Blood pressure calculation method
2.3. Appearance example of the measurement apparatus
3. Second embodiment of the present disclosure
4. Calculation method processing procedure
5. Modified examples of the first and second embodiments of the present disclosure
5.1. Modified example of the chest contact measurement unit
5.2. Modified example of the configuration of the measurement apparatus
5.3. Modified example of the usage method of the measurement apparatus
5.4. Other modified examples
6. Measurement apparatus hardware configuration

### 7. Summary

### 1. Investigations by the Inventor

Before providing a detailed description of the embodiments of the present disclosure, a detailed description will be made regarding the results of an investigation performed by the inventor regarding a typical pulse wave system blood pressure monitor.

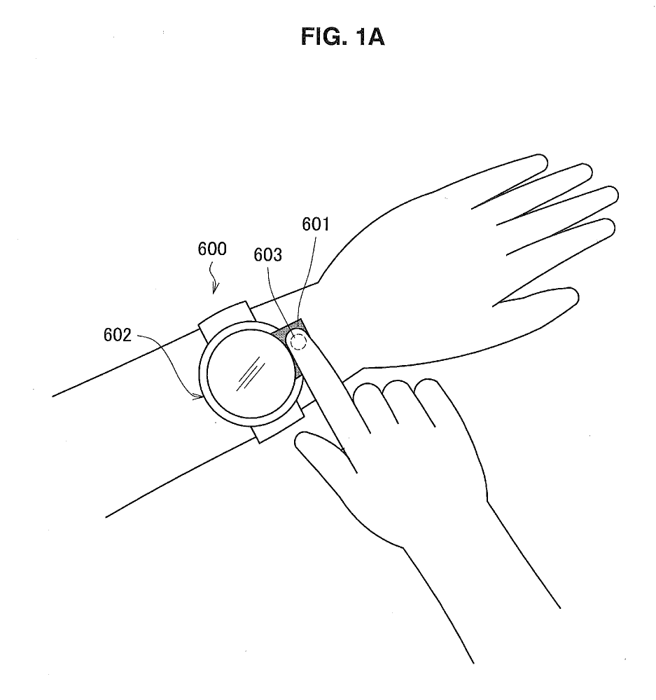

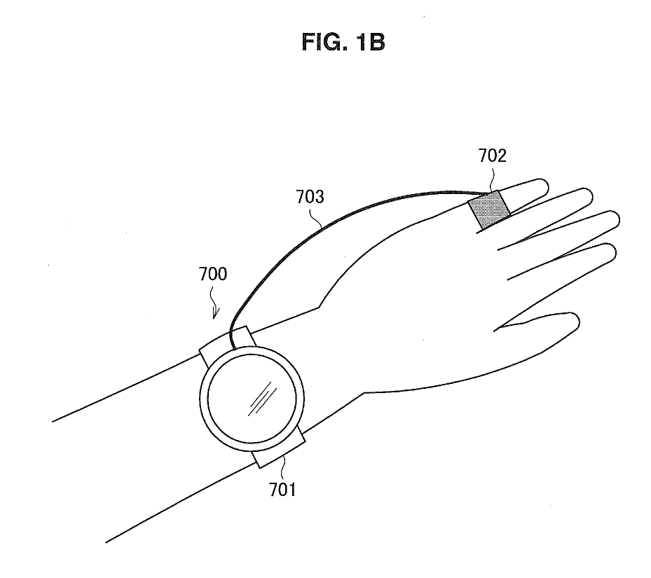

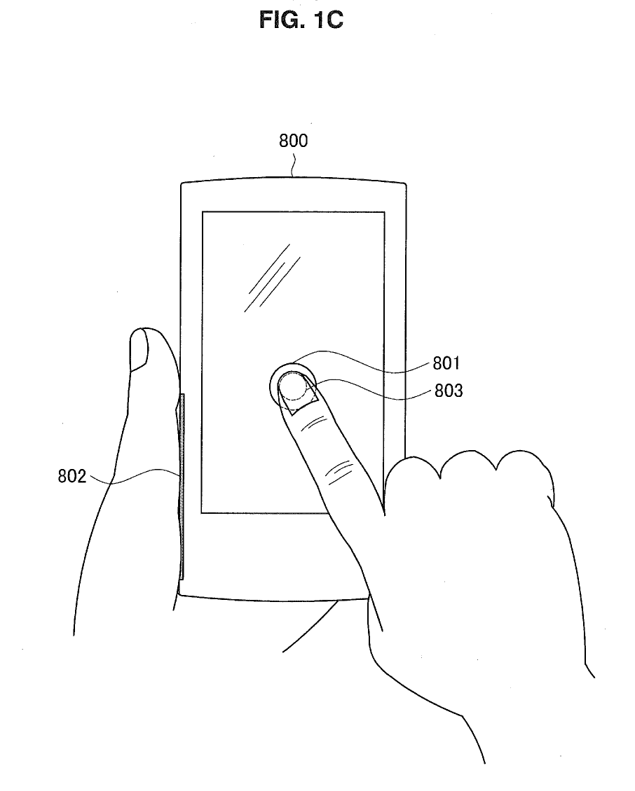

FIG. 1A is a schematic diagram illustrating a configuration example of the pulse wave system blood pressure monitor described in the above JP-A-2002-172094. As illustrated in FIG. 1A, a pulse wave system blood pressure monitor 600 is a wristwatch type blood pressure monitor that is provided with a first electrode 601 at a position on the flank of the dial and a pulse wave measurement detection window 603. Further, a second electrode 602 is provided at a position in contact with the arm, which corresponds to the back of the dial. Here, the pulse wave measurement detection window 603 is an optical sensing unit for detecting a pulse wave using light. When measuring blood pressure, the measurement subject simultaneously touches the first electrode 601 and the pulse wave measurement detection window 603 with, for example, a finger on his/her hand on which the blood pressure monitor 600 is not worn. Since the second electrode 602 is in contact with the arm on which the wristwatch type blood pressure monitor 600 is worn, electrocardiography-related data is measured from the difference in potential between the two electrodes. On the other hand, pulse wave-related data is measured from the finger touching the pulse wave measurement detection window 603.

As described above, in the pulse wave system blood pressure monitor 600 illustrated in FIG. 1A, electrocardiography-related data is measured as the difference in potential between the finger and the arm of the measurement subject. However, when the electrocardiography-related data is measured between the measurement subject's finger and arm, since there are individual differences in the position and angle of a person's heart, depending on the measurement subject, the electrocardiography-related data may not be accurately measured. Further, due to loosening of the belt wrapped about the arm or the like, contact between the second electrode 602 and the arm can become unstable, which can prevent the measurement from being performed accurately.

FIG. 1B is a schematic diagram illustrating a configuration example of the pulse wave system blood pressure monitor described in the above JP-A-2004-201868. As illustrated in FIG. 1B, a pulse wave system blood pressure monitor 700 is a wristwatch type blood pressure monitor that includes a first pulse wave measurement unit 701 at a position corresponding to a belt. Further, the pulse wave system blood pressure monitor 700 also includes a second pulse wave measurement unit 702 that can be worn on any finger. In addition, the second pulse wave measurement unit 702 is connected by a cable 703 to a main body of the pulse wave system blood pressure monitor 700. The blood pressure monitor 700 can calculate blood pressure by determining a pulse wave transit time (velocity) from the time difference between a pulse wave at the first pulse wave measurement unit 701 and a pulse wave at the second pulse wave measurement unit 702.

As described above, with the pulse wave system blood pressure monitor 700 illustrated in FIG. 1B, if the subject wants to constantly measure blood pressure, the subject has to constantly fit the second pulse wave measurement unit 702 to his/her finger. Further, since the cable 703 is provided, from the perspective of user friendliness for the subject, it is difficult to continuously wear on a daily basis. Moreover, similar to the pulse wave system blood pressure monitor 600 illustrated in FIG. 1A, due to loosening of the belt wrapped about the arm or the like, the pulse wave measurement by the first pulse wave measurement unit 701 may not be performed accurately.

FIG. 1C is a schematic diagram illustrating a configuration example of a so-called handy-type pulse wave system blood pressure monitor. As illustrated in FIG. 1C, a pulse wave system blood pressure monitor 800 has a panel-like shape. A first electrode 801 and a pulse wave measurement detection window 803 are provided on a partial area on one face of the panel. Further, a second electrode 802 is provided on a partial area on a side face of the panel. Here, the pulse wave measurement detection window 803 is an optical sensing unit for detecting a pulse wave using light. When measuring blood pressure, the measurement subject touches the second electrode 802 with, for example, the hand that is holding the blood pressure monitor 800. Further, the measurement subject simultaneously touches the first electrode 801 and the pulse wave measurement detection window 803 with a finger on his/her hand on which the blood pressure monitor 800 is not being held. Electrocardiography-related data is measured from the difference in potential between the two electrodes, and pulse wave-related data is measured from the finger touching the pulse wave measurement detection window 803.

As described above, with the pulse wave system blood pressure monitor 800 illustrated in FIG. 1C, electrocardiography-related data is measured as the difference in potential between the finger and the hand of the measurement subject. Therefore, similar to the pulse wave system blood pressure monitor 600 illustrated in FIG. 1A, depending on the measurement subject, the electrocardiography-related data may not be accurately measured. Further, since the blood pressure monitor 800 has a panel-like shape, this blood pressure monitor 800 is not suited to usage methods in which the monitor is casually carried around or in which blood pressure is constantly measured.

As described above, with a typical pulse wave system blood pressure monitor, depending on the measurement subject, the electrocardiography-related data may not be accurately measured. Further, a typical pulse wave system blood pressure monitor is not suited to being used on a daily basis, or to being used while casually carried around.

Thus, for a typical pulse wave system blood pressure monitor, there is room for improvement in the accuracy of the measured blood pressure and in user convenience. Accordingly, based on the results of investigations concerning a measurement apparatus capable of realizing a more accurate blood pressure measurement and better user convenience, the present inventor conceived of the measurement apparatus, measurement method, program, storage medium, and measurement system that are described below in detail.

### 2. First Embodiment of the Present Disclosure>

### 2.1. Configuration of the Measurement Apparatus

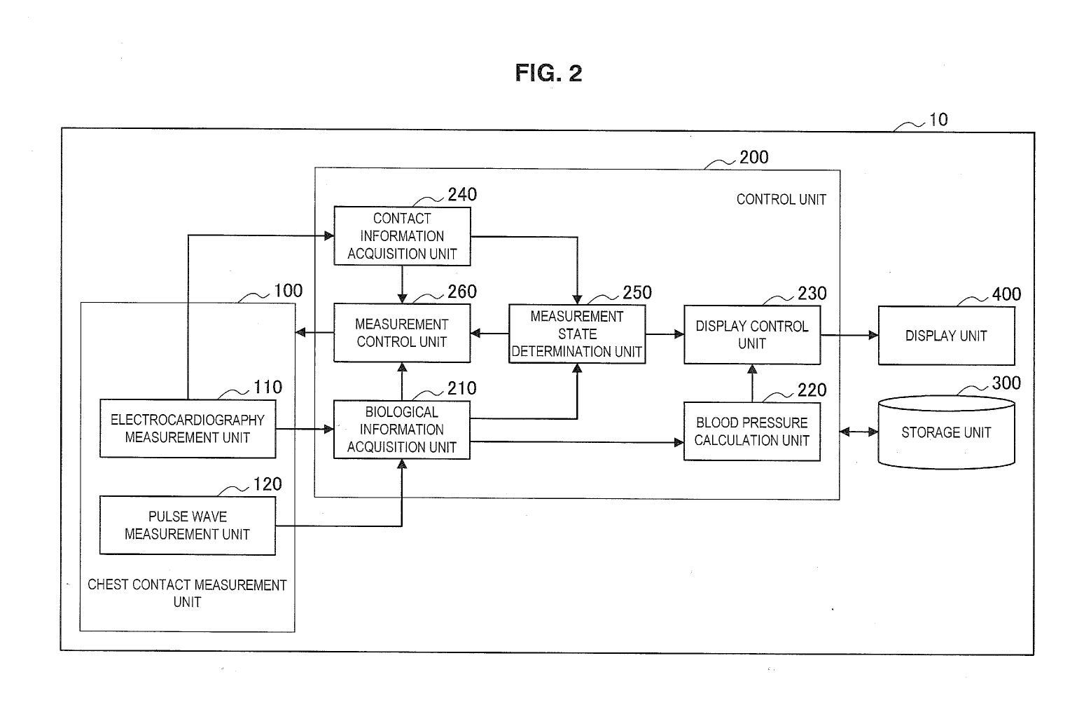

First, a schematic configuration of a measurement apparatus according to a first embodiment of the present disclosure will be described with reference to FIG. 2. FIG. 2 is a function block diagram illustrating a schematic configuration of a measurement apparatus according to a first embodiment of the present disclosure.

As illustrated in FIG. 2, a measurement apparatus 10 according to this embodiment of the present disclosure includes a chest contact measurement unit 100, a control unit 200, a storage unit 300, and a display unit 400.

The chest contact measurement unit 100 measures various types of data relating to biological activity of a measurement subject. Here, the various types of data (biological information) relating to biological activity may be, for example, data (information) relating to electrocardiography (electrocardiogram), pulse rate, heart rate, breathing, body temperature and the like. The chest contact measurement unit 100 has, for example, an electrocardiography measurement unit 110 and a pulse wave measurement unit 120.

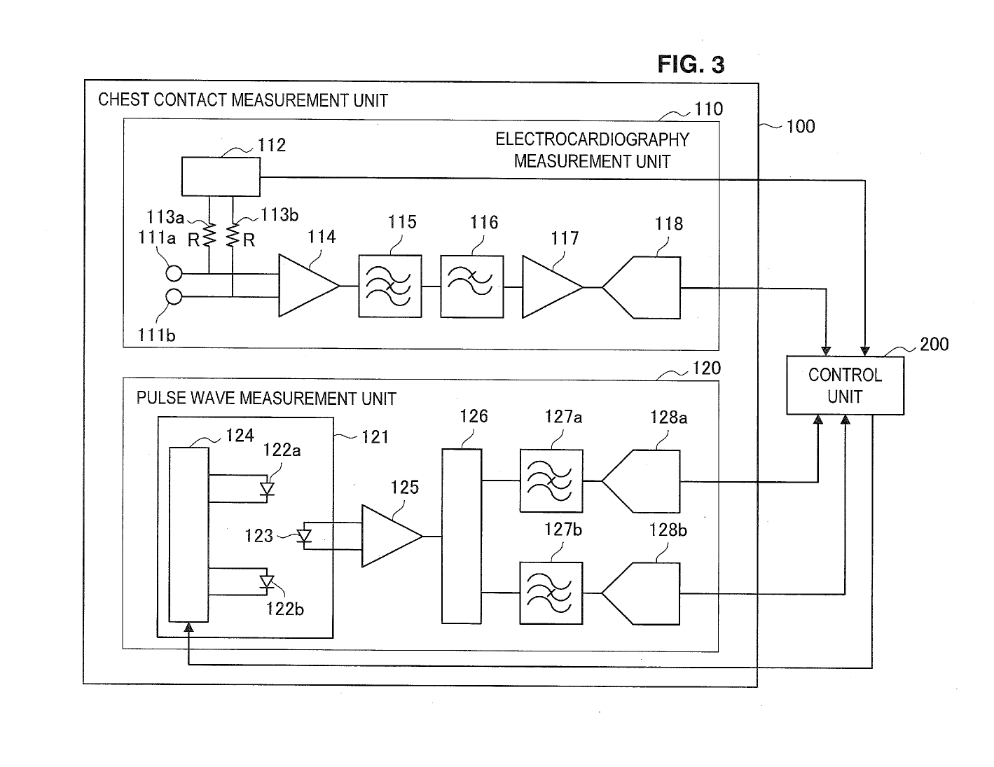

The electrocardiography measurement unit 110 is brought into contact with the measurement subject's chest to measure electrocardiography-related data of the measurement subject from a chest measurement site. The pulse wave measurement unit 120 measures pulse wave-related data of the measurement subject from a pulse wave detection site of the measurement subject. Here, the electrocardiography-related data (electrocardiography information) may include, for example, data (information) relating to the heart rate, the electrocardiography waveform, an electrocardiogram and the like of the measurement subject. Further, the pulse wave-related data (pulse wave information) may include data (information) relating to the pulse rate, pulse wave, blood flow and the like at a pulse wave detection site. Further, in the following description, electrocardiography measurement refers to the measurement of electrocardiography-related data of the measurement subject, and pulse wave measurement refers to the measurement of pulse wave-related data of the measurement subject. The configuration of the electrocardiography measurement unit 110 and the pulse wave measurement unit 120 will be described in detail below with reference to FIG. 3.

The control unit 200 controls the measurement apparatus 10 in an integrated manner, and, for example, processes the various types of data relating to biological activity measured by the chest contact measurement unit 100. Specifically, the control unit 200 calculates the measurement subject's blood pressure based on the various types of data relating to biological activity measured by the chest contact measurement unit 100. Further, the control unit 200 controls measurement performed by the chest contact measurement unit 100 based on information relating to a contact state (contact information) between the chest contact measurement unit 100 and the chest measurement site of the measurement subject. In addition, the control unit 200 determines the reliability of the various types of data relating to biological activity measured by the chest contact measurement unit 100. Still further, the control unit 200 controls the display on the display unit 400 of the results obtained by processing the various types of data relating to biological activity.

The function and the configuration of the control unit 200 will now be described in more detail. The control unit 200 has a biological information acquisition unit 210, a blood pressure calculation unit 220, a display control unit 230, a contact information acquisition unit 240, a measurement state determination unit 250, and a measurement control unit 260.

The biological information acquisition unit 210 acquires the various types of data relating to biological activity, namely, biological information, measured by the chest contact measurement unit 100. For example, the biological information acquisition unit 210 acquires electrocardiography-related data, namely, electrocardiography information, of the measurement subject measured by the electrocardiography measurement unit 110. Further, the biological information acquisition unit 210 may also acquire pulse wave-related measurement data, namely, pulse wave information, of the measurement subject measured by the pulse wave measurement unit 120. In addition, the biological information acquisition unit 210 transmits the acquired biological information to the blood pressure calculation unit 220, the measurement state determination unit 250, and the measurement control unit 260.

The blood pressure calculation unit 220 calculates a blood pressure value of the measurement subject based on the biological information transmitted from the biological information acquisition unit 210. For example, the blood pressure calculation unit 220 calculates a pulse wave transit time (velocity) of the measurement subject based on the electrocardiography information and the pulse wave information for the measurement subject, and based on the calculated pulse wave transit time (velocity), calculates the blood pressure value. The specific method for calculating the blood pressure will be described in detail in (2.2 Blood pressure calculation method). Further, the blood pressure calculation unit 220 transmits the calculated information relating to the blood pressure value to the display control unit 230, for example.

The display control unit 230 controls the display on the display unit 400 of the various types of information processed by the control unit 200 and the results obtained by processing these pieces of information. For example, the display control unit 230 controls the display on the display unit 400 of the blood pressure value calculated by the blood pressure calculation unit 220. Further, the display control unit 230 can also receive biological information about the measurement subject, for example electrocardiography information, pulse wave information and the like, from the blood pressure calculation unit 220, and control the display of this biological information on the display unit 400. In addition, the display control unit 230 can also control the display on the display unit 400 of information relating to the reliability of the biological information for the measurement subject determined by the below-described measurement state determination unit 250. Moreover, the display control unit 230 can control the display on the display unit 400 of a guide or the like relating to calibration performed in a below-described blood pressure calculation method.

The contact information acquisition unit 240 acquires contact information, which is information relating to the contact state between the chest contact measurement unit 100 and the chest measurement site of the measurement subject. Here, the contact information may also be information relating to the contact state between the electrocardiography measurement unit 110 and the chest measurement site of the measurement subject. Further, the contact information may include information relating to the presence/absence of contact between the electrocardiography measurement unit 110 and the chest measurement site of the measurement subject. In addition, the contact information may also include information relating to the duration of contact between the electrocardiography measurement unit 110 and the chest measurement site of the measurement subject. Still further, the contact information may also include information relating to the strength with which the electrocardiography measurement unit 110 presses against the chest measurement site of the measurement subject. The contact information acquisition unit 240 transmits the acquired contact information to the measurement state determination unit 250 and the measurement control unit 260.

The measurement state determination unit 250 determines the reliability of the various types of data relating to biological activity measured by the chest contact measurement unit 100 based on the received biological information and the contact information. Specifically, the measurement state determination unit 250 may determine whether the electrocardiography-related data of the measurement subject has been appropriately measured based on, for example, the strength, which is included in the contact information, with which the electrocardiography measurement unit 110 presses against the chest measurement site of the measurement subject, by determining whether that pressing strength is greater than a pre-set threshold. The method used to measure the strength with which the electrocardiography measurement unit 110 is being pressed will be described below in more detail with reference to FIG. 3.

Here, the pulse wave measurement unit 120 is provided on, for example, a face that opposes the contact face between the electrocardiography measurement unit 110 and the chest with respect to the chest contact measurement unit 100. If the electrocardiography measurement unit 110 and the pulse wave measurement unit 120 have the above-described positional relationship, by holding the chest contact measurement unit 100 in front of the measurement subject's chest and bringing the pulse wave detection site of the measurement subject into contact with or to press against the pulse wave measurement unit 120, the electrocardiography measurement unit 110 is brought into contact with or made to press against the chest measurement site of the measurement subject. Therefore, the contact information can indirectly include information relating to the contact state (contact presence/absence, pressing strength etc.) between the pulse wave detection site and the pulse wave measurement unit 120.

If the contact information includes information relating to the contact state between the pulse wave detection site and the pulse wave measurement unit 120, the measurement state determination unit 250 may determine whether the pulse wave-related data of the measurement subject has been appropriately measured based on the strength, which is included in the contact information, with which the electrocardiography measurement unit 110 presses against the chest measurement site of the measurement subject, for example, by determining whether that strength is greater than a pre-set threshold. Here, although the above-described threshold used as a determination reference by the measurement state determination unit 250 is not especially limited, the threshold can be appropriately set based on, for example, past measurement data or the like that has been statistically acquired. Further, these thresholds may also be freely set by the measurement subject before measurement starts.

Further, the measurement state determination unit 250 can determine the reliability of these pieces of data by comparing, other than contact information, the measured electrocardiography-related data and/or the pulse wave-related data of the measurement subject with previously measured data. For example, if the measured electrocardiography-related data and/or the pulse wave-related data of the measurement subject is greatly different from the previously measured data, the measurement state determination unit 250 can determine that measurement was not performed appropriately.

The measurement state determination unit 250 transmits the determined information relating to the reliability of the biological information about the measurement subject to the display control unit 230 and the measurement control unit 260.

The measurement control unit 260 controls the measurement of the various types of data relating to biological activity by the chest contact measurement unit 100. Specifically, the measurement control unit 260 may control the electrocardiography measurement unit 110 so that measurement of the electrocardiography-related data is started when the electrocardiography measurement unit 110 has contacted the chest measurement site, for example, based on contact information received from the contact information acquisition unit 240. Further, if measurement by the electrocardiography measurement unit 110 is thus started based on contact information, the measurement apparatus 10 may be in a power standby state (power saving state) until contact between the electrocardiography measurement unit 110 and the chest measurement site is detected. Here, power standby state refers to a state in which the units other than the unit for detecting contact between the electrocardiography measurement unit 110 and the chest measurement site are not functioning (are powered off). The control for waking up the measurement apparatus 10 from a power standby state and the control for making the measurement apparatus 10 enter a power standby state can be performed by the control unit 200, for example.

In addition, the measurement control unit 260 can also control the electrocardiography measurement unit 110 based on the contact information so that measurement of the electrocardiography-related data is finished when the electrocardiography measurement unit 110 and the chest measurement site are separated. If measurement by the electrocardiography measurement unit 110 is thus finished based on contact information, the measurement apparatus 10 may be configured so as to enter a power standby state when the electrocardiography measurement unit 110 and the chest measurement site separate.

Still further, the measurement control unit 260 can control measurement by the chest contact measurement unit 100 based on information relating to the reliability of the biological information received from the measurement state determination unit 250. For example, the measurement control unit 260 can control the electrocardiography measurement unit 110 so that measurement is finished if it is determined that electrocardiography measurement by the electrocardiography measurement unit 110 was appropriately performed, and measurement is performed again if it is determined that electrocardiography measurement was not appropriately performed. In addition, for example, the measurement control unit 260 can control the electrocardiography measurement unit 110 so that, similarly, measurement is finished if it is determined that pulse wave measurement by the pulse wave measurement unit 120 was appropriately performed, and measurement is performed again if it is determined that pulse wave measurement was not appropriately performed.

In the above, a schematic configuration of the control unit 200 according to this embodiment of the present disclosure was described in detail. Here, the configuration of the control unit 200 is not limited to the example illustrated in FIG. 2. As long as the configuration fulfills the above-described functions, the control unit 200 may be configured from any function block.

Next, the storage unit 300 according to this embodiment of the present disclosure will be described. The storage unit 300 stores the various types of data relating to biological activity measured by the chest contact measurement unit 100 and/or various types of data processed by the control unit 200. The storage unit 300 stores, for example, electrocardiography-related data, pulse wave-related data, and contact information measured by the chest contact measurement unit 100. Further, the storage unit 300 can also store information relating to the blood pressure value calculated by the blood pressure calculation unit 220 and/or information relating to the reliability of the biological information determined by the measurement state determination unit 250.

Further, the storage unit 300 can also store information relating to a calculation formula, parameters (functions), constant values and the like used in the below-described blood pressure calculation method. The blood pressure calculation unit 220 cam calculate the blood pressure value by referring to the calculation formula, parameters (functions), constant values and the like that are stored in the storage unit 300.

In addition, in FIG. 2, although an example is illustrated in which the storage unit 300 is provided in the measurement apparatus 10, this embodiment of the present disclosure is not limited to this. For example, the measurement apparatus 10 may further include a (not illustrated) connection port for external device connection, and be connected to a (not illustrated) externally-provided external storage unit via this connection port. If the measurement apparatus 10 is connected to an external storage unit, the above-described various types of data that can be stored in the storage unit 300 may be stored in the external storage unit. Here, for example, the connection port may be a memory card connector, and the external storage unit may be a memory card.

The display unit 400 displays the various types of information processed by the control unit 200 under the control of the display control unit 230. The display unit 400 may be a display, for example. If the display unit 400 is a display, the display unit 400 can display the measured electrocardiography-related data and pulse wave-related data, and the calculated blood pressure value and the like in numerical or graphical form, for example. Further, the contact state between the electrocardiography measurement unit 110 and the chest measurement site, and the information relating to the reliability of the measured biological activity-related data (whether the data relating to biological activity was appropriately measured) may be displayed on the display as characters, numerals, symbols and the like.

Further, the display unit 400 may also be a light-emitting diode (LED). If the display unit 400 is a LED, for example, the LED can be made to flash in synchronization with the period of the electrocardiography waveform included in the electrocardiography information or the period of the pulse wave included in the pulse wave information. In addition, the LED can be turned off when the measurement apparatus 10 is in a power standby state, and turned on when the measurement apparatus 10 is not in a power standby state. Still further, based on the information relating to the reliability of the measured biological activity-related data, if it is determined that, for example, the electrocardiography-related data was not appropriately measured, a warning of that fact can be displayed by, for example, turning on an LED having a different color.

In addition, the measurement apparatus 10 may further include a (not illustrated) audio output unit configured from, for example, a speaker, a headphone or the like. If the measurement apparatus 10 includes an audio output unit, various reminders, warnings and the like performed by the above-described display unit 400 LED may be realized by the audio output unit outputting an alarm sound or the like.

Still further, the measurement apparatus 10 according to this embodiment of the present disclosure may further include a (not illustrated) communication unit for transmitting and receiving information to and from various external devices. For example, the communication unit may transmit the various types of data relating to biological activity measured by the chest contact measurement unit 100 and/or various types of data processed by the control unit 200 to an external device. Specifically, the communication unit may transmit electrocardiography-related data and/or pulse wave-related data measured by the chest contact measurement unit 100 to an external device. Further, the communication unit may transmit information relating to the calculated blood pressure value to an external device. In addition, the communication unit may transmit information relating to the contact state between the electrocardiography measurement unit 110 and the chest measurement site, and information relating to the reliability of the measured data relating to biological activity to an external device. Here, the timing at which the communication unit transmits the various types of data to an external unit may be, for example, in real time each time the various types of data relating to biological activity are measured, or the various types of data may be transmitted collectively after a series of blood pressure measurement processes has finished.

Moreover, the communication unit can also receive information relating to a calculation formula, parameters (functions), constant values and the like used in a below-described blood pressure calculation method, and store the received information in the storage unit 300 or in the above-mentioned external storage unit. Therefore, these calculation formula, parameters (functions), constant values and the like that can be updated via the communication unit.

Further, various communication methods, either wired or wireless, may be employed as the communication method that the communication unit employs. If the communication unit has a wireless transmission function, the wireless transmission method that is used may be, for example, Bluetooth® (IEEE 802.15.1), which is a near field communication system, or IEEE 802.15.6, which has been standardized as body area network.

In addition, the external device that communicates to and from the measurement apparatus 10 via the communication unit may be a PC (personal computer), or a server. Further, these external devices may be configured so as to have the same functions as the control unit 200. If an external device does have the same function as the control unit 200, then that external device can perform the same calculation processing as performed by the control unit 200 on the various types of data transmitted from the measurement apparatus 10.

Next, a schematic configuration of the electrocardiography measurement unit 110 and the pulse wave measurement unit 120 included in the chest contact measurement unit 100 will be described with reference to FIG. 3. FIG. 3 is a function block diagram illustrating a schematic configuration of the chest contact measurement unit 100 illustrated in FIG. 2.

As illustrated in FIG. 3, the electrocardiography measurement unit 110 is configured from, for example, electrodes 111a and 111b, a skin resistance detection device 112, shunt resistors 113a and 113b, a difference amplifier 114, a notch filter 115, a low-pass filter 116, an amplifier 117, and an analog-to-digital converter (AD converter) 118. In the following, the skin resistance detection device 112 will be described as an example of the contact detection unit for measuring data relating to contact information. However, the contact detection unit may be some other configuration, as long as it can measure data relating to contact information.

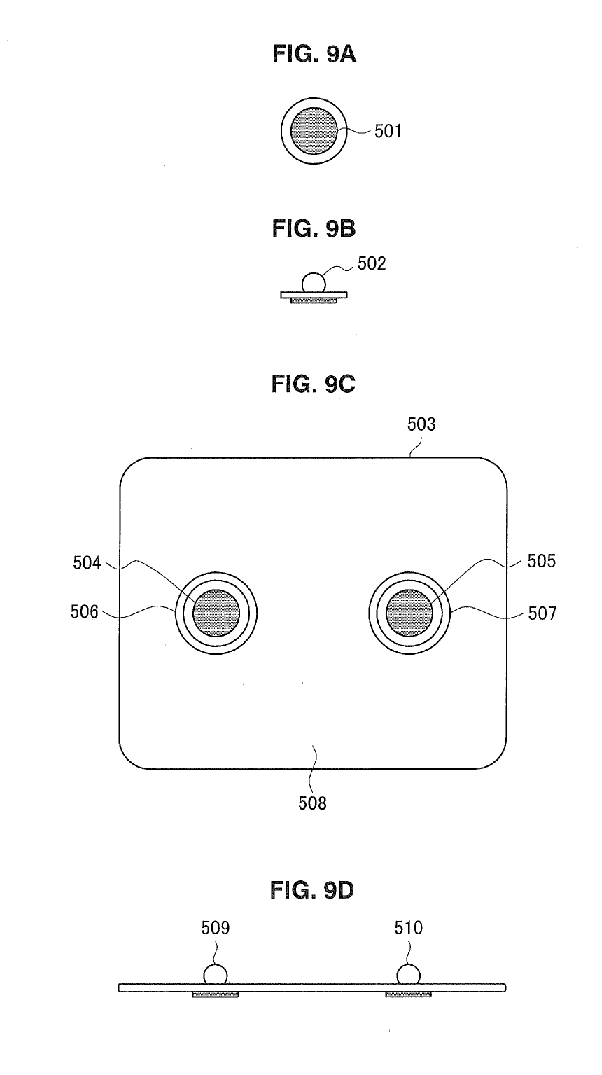

The electrodes 111a and 111b are brought into contact with the chest measurement site of the measurement subject, and the difference in potential between the two electrodes is measured. Since the electrocardiography measurement is carried out by measuring the difference in potential between two desired points on the body, the difference in potential between electrode 111a and electrode 111b corresponds to the electrocardiography-related data (signal). Further, by measuring the change over time in this difference in potential, information relating to the electrocardiography waveform can be obtained. The configuration of the electrodes 111a and 111b will be described below in detail with reference to FIG. 9.

The skin resistance detection device 112 detects the direct current resistance between the electrode 111a and electrode 111b, and transmits the detected resistance to the contact information acquisition unit 240 of the control unit 200. When the electrodes 111a and 111b are in contact with the skin, a minute current flows between the electrodes via the skin. The resistance value when the electrodes 111a and 111b are in contact with the skin is a value around, for example, several hundred kΩ to several MΩ. Therefore, based on the skin resistance value detected by the skin resistance detection device 112, information about whether the electrodes 111a and 111b and the chest measurement site are in contact, namely, information relating to the presence/absence of contact between the electrocardiography measurement unit 110 and the chest measurement site, is obtained. Further, the resistance value between the two electrodes also changes based on the strength with which the electrodes 111a and 111b are pressing on the chest measurement site. Therefore, based on the skin resistance value detected by the skin resistance detection device 112, information relating to the strength with which the electrocardiography measurement unit 110 is being pressed on the chest measurement site can be obtained.

The shunt resistors 113a and 113b play the role of protecting the circuit from eddy currents that are produced when the electrode 111a and the electrode 111b short. The resistance value of the shunt resistors 113a and 113b may be appropriately designed based on the design value of a common shunt resistor and shunt circuit.

The difference amplifier 114 amplifies the difference in potential between the electrode 111a and the electrode 111b. Generally, since the difference in potential between the electrode 111a and the electrode 111b is about a few mV, the difference amplifier 114 is designed so that this difference in potential is amplified by about 100-fold, for example.

The notch filter 115 is a filter for removing unwanted noise from the signal amplified by the difference amplifier 114. The notch filter 115 is a filter circuit for reducing the frequency component of a specific band. In this embodiment of the present disclosure, for example, in consideration of the effects from commercial alternating current power sources present near the electrocardiography measurement unit 110, the notch filter 115 is designed so as to reduce the bands near 50 Hz or 60 Hz. Further, the low-pass filter 116 is a filter circuit for removing wideband noise that is unwanted in electrocardiography measurement. In this embodiment of the present disclosure, for example, in consideration of the fact that the frequency of an electrocardiography waveform is about several Hz, the cutoff frequency is set at around 100 Hz.

Here, since the removal of unwanted signals is performed as necessary even during the subsequent signal processing steps (signal processing steps performed by the control unit 200), the properties of the notch filter 115 and the low-pass filter 116 may be freely designed, as long as those properties allow the filters to remove signals at a level at which they are not overwhelmed by the amplification system.

The amplifier 117 amplifies the signal in which unwanted noise has been reduced by the notch filter 115 and the low-pass filter 116. The amplifier 117 gain is, for example, set at about 10 times. Therefore, for example, the difference in potential between the electrode 111a and the electrode 111b, which was about several mV, is ultimately amplified to about several hundred mV to 1 V, and input into the AD converter 118.

The AD converter 118 converts (analog to digital conversion) the input signal, namely, the amplified electrocardiography-related signal, from an analog signal into a digital signal, and transmits the converted digital signal to the biological information acquisition unit 210 of the control unit 200.

Next, a schematic configuration of the pulse wave measurement unit 120 will be described. As illustrated in FIG. 3, the pulse wave measurement unit 120 is configured from, for example, an optical sensing unit 121, an amplifier 125, a multiplexer 126, band-pass filters 127a and 127b, and AD converters 128a and 128b.

The optical sensing unit 121 performs optical measurement for measuring a pulse wave at the pulse wave detection site. The optical sensing unit 121 is configured from, for example, LEDs 122a and 122b, a photodiode 123, and a drive unit 124.

Here, the principles of pulse wave measurement will now be described. Generally, the hemoglobin present in blood tends to absorb light having a specific wavelength. Since the amount of hemoglobin is proportional to the blood flow in a blood vessel, when light having a specific wavelength is irradiated on a pulse wave detection site and light that has passed through or been reflected therefrom is detected, the amount of light that is detected also changes based on the blood flow in the blood vessel. Therefore, changes in the blood flow in a blood vessel can be measured from the detected amount of light, which allows a pulse wave to be measured.

Further, the light absorption spectrum of hemoglobin is different for hemoglobin bound to oxygen and hemoglobin that is not bound to oxygen. For example, for infrared light (e.g., wavelength of about 940 nm) the effect on light absorbance due to changes in arterial oxygen saturation (SpO2) is comparatively small. On the other hand, for red light (e.g., wavelength of about 660 nm), the effect on light absorbance due to changes in arterial oxygen saturation is comparatively large. Here, arterial oxygen saturation is an index indicating the ratio of hemoglobin bound to oxygen in arterial blood.

Therefore, utilizing the wavelength dependence of light absorbance based on the presence or absence of hemoglobin-bound oxygen, arterial oxygen saturation can be measured simultaneously with pulse wave measurement by irradiating two types of light, infrared light and red light, on a pulse wave detection site. An embodiment of the present disclosure in which arterial oxygen saturation is measured simultaneously with a pulse wave will bow be described.

Returning to the description of the optical sensing unit 121, the LEDs 122a and 122b are light-emitting elements that, as described above, emit infrared light having a wavelength of about 940 nm and red light having a wavelength of about 660 nm, respectively. The LEDs 122a and 122b, which are controlled by the below-described drive unit 124, alternately irradiate light in the respective wavelengths on the pulse wave detection site, for example.

The photodiode 123, which is a light receiving element, detects light that has passed through or that has been reflected from the pulse wave detection site from among the light irradiated from the LED 122a or LED 122b, and inputs a signal that is based on the received light amount into the amplifier 125. Here, for example, if the photodiode 123 is detecting light that passes through the pulse wave detection site, the LEDs 122a and 122b and the photodiode 123 are arranged so as to sandwich the pulse wave detection site. Further, for example, if the photodiode 123 is detecting light that is reflected from the pulse wave detection site, the LEDs 122a and 122b and the photodiode 123 are arranged on the same side as each other with respect to the pulse wave detection site.

The drive unit 124 controls the drive of the LEDs 122a and 122b based on, for example, a control from the control unit 200. Specifically, the drive unit 124 may control so that the LEDs 122a and 122b alternately emit light at fixed intervals.

The amplifier 125 amplifies the electric signal input from the photodiode 123, and inputs the amplified signal into the multiplexer 126. The gain of the amplifier 125 may be appropriately designed based on the light amount of the LEDs 122a and 122b, for example.

The multiplexer 126 selects either the band-pass filter 127a or band-pass filter 127b based on the wavelength of the light emitted by the LEDs 122a and 122b, and inputs the signal amplified by the amplifier 125. Here, the band-pass filters 127a and 127b are set so as to reduce the frequency component of the bands other than infrared or red light, for example. Therefore, if the light detected by the photodiode 123 is transmitted light or reflected light from the LED 122a that emits infrared light, the multiplexer 126 selects the band-pass filter 127a that is set to correspond to infrared light, and outputs the signal. Further, if the light detected by the photodiode 123 is transmitted light or reflected light from the LED 122b that emits red light, the multiplexer 126 selects the band-pass filter 127b that is set to correspond to red light, and outputs the signal.

The noise-reduced signal that has passed through the band-pass filters 127a and 127b is input into the AD converters 128a and 128b, respectively. Since the function of the AD converters 128a and 128b is the same as that of the AD converter 118, a detailed description will be omitted here. The respective digitalized signals corresponding to the infrared light and red light each form a pulse wave signal. The AD converters 128a and 128b transmit the digitally-converted signals to the biological information acquisition unit 210 of the control unit 200.

Thus, the biological information acquisition unit 210 can acquire pulse wave-related data from the transmitted light or reflected light detected when infrared light and/or red light is irradiated on the pulse wave detection site. Further, the biological information acquisition unit 210 can acquire data relating to arterial oxygen saturation from the transmitted light or reflected light detected when infrared light and red light are alternately irradiated on the pulse wave detection site.

As described above, in the measurement apparatus 10 according to the first embodiment of the present disclosure, electrocardiography-related data is measured by the electrocardiography measurement unit 110 that has been brought into contact with the chest, and pulse wave-related data is measured by the pulse wave measurement unit 120. Further, the blood pressure calculation unit 220 calculates the blood pressure value of the measurement subject based on that electrocardiography information and pulse wave information. By having the above configuration, since electrocardiography measurement is performed at a chest measurement site, the electrocardiography measurement and the blood pressure measurement can be carried out more accurately.

Further, in the measurement apparatus 10 according to the first embodiment of the present disclosure, the contact information acquisition unit 240 acquires contact information, which is information relating to the contact state between the electrocardiography measurement unit 110 and the chest measurement site. In addition, based on at least the contact information, the measurement state determination unit 250 determines at least either the reliability of the electrocardiography information or the reliability of the pulse wave information. By having the above configuration, if the reliability of the electrocardiography information and/or the pulse wave information is low, the accuracy of the electrocardiography measurement and/or pulse wave measurement can be improved by appropriately adjusting the contact state (contact position, pressing strength etc.) between the electrocardiography measurement unit 110 and the chest measurement site. Therefore, the electrocardiography measurement and pulse wave measurement can be carried out more accurately.

In the above, an example of the functions of the measurement apparatus 10 according to this embodiment of the present disclosure, especially an example of the functions of the chest contact measurement unit 100 and the control unit 200, was described with reference to FIGS. 2 and 3. Here, the above-described constituent elements may be configured using versatile parts and circuits, or may be configured from hardware that is specialized to the function of each constituent element. Further, regarding the control unit 200, the functions of the respective constituent element may all be performed by a CPU (central processing unit) and the like. Therefore, the configuration that is utilized can be appropriately changed based on the technological level at the time when this embodiment of the present disclosure is worked.

Further, although the circuit configuration of the electrocardiography measurement unit 110 and the pulse wave measurement unit 120 were described in detail with reference to FIG. 3, these circuit configurations are not limited to the illustrated example. The circuit configuration of the electrocardiography measurement unit 110 and the pulse wave measurement unit 120 can be appropriately changed as long as the above-described desired functions can be realized. For example, in the above-described circuit configuration example, although a case was described in which the pulse wave measurement unit 120 simultaneously measures the pulse wave and the arterial oxygen saturation of the measurement subject, the pulse wave measurement unit 120 may perform only pulse wave measurement. If the pulse wave measurement unit 120 performs only pulse wave measurement, the optical sensing unit 121 is configured having only one LED, and the configuration of the subsequent multiplexer 126, band-pass filters 127a and 127b, and AD converters 128a and 128b can also be appropriately changed.

### 2.2. Blood Pressure Calculation Method

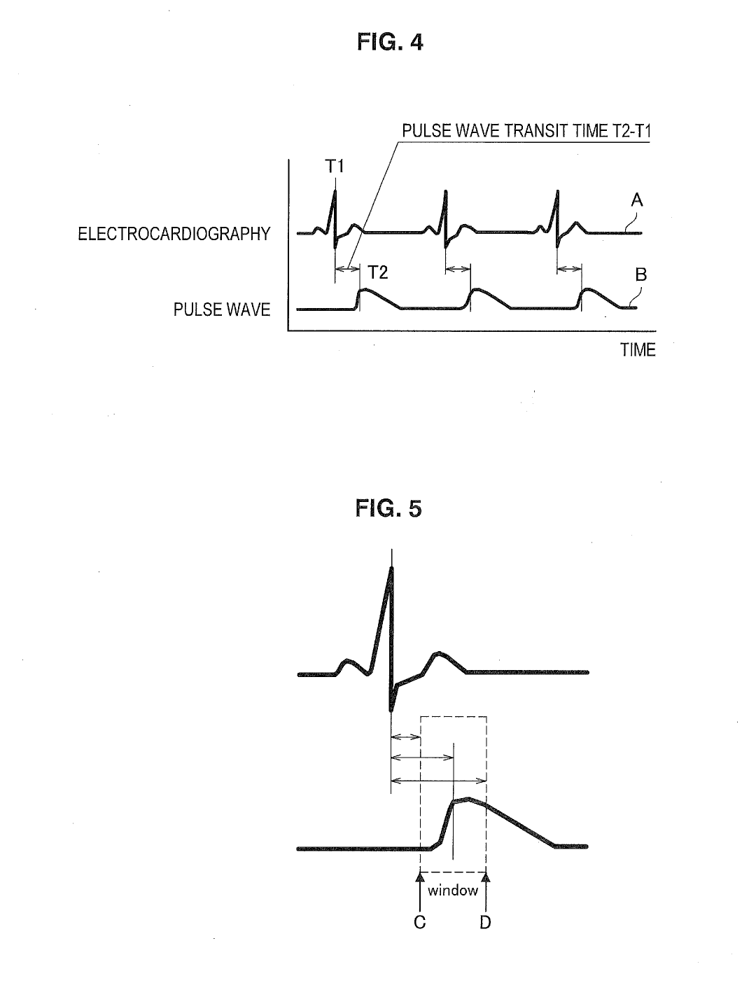

The method for calculating blood pressure with the measurement apparatus 10 according to the first embodiment of the present disclosure will now be described with reference to FIGS. 4 to 7.

First, the method for calculating the pulse wave velocity (time) will be described with reference to FIGS. 4 and 5. FIG. 4 is a graph that plots an electrocardiography waveform and a pulse wave. FIG. 5 is an expanded diagram illustrating a period close to time T1 and time T2 in FIG. 4 in an expanded manner.

As illustrated in FIG. 4, the change over time in the signal intensity of an electrocardiography waveform A and the change over time in the signal intensity of a pulse wave B are plotted on a plane formed from a horizontal axis representing time and a vertical axis representing signal intensity. Here the pulse wave B is the pulse wave measured at the finger tip of the measurement subject, for example.

In the periodic waveform of electrocardiography waveform A, for example, if the time at the initial rise of a given wave (R wave) is T1, and in the periodic waveform of pulse wave B, the time at the initial rise of a given wave that appears after time T1 is T2, then the pulse wave transit time can be defined as T2-T1. Further, the pulse wave velocity can be defined as, for example, the value obtained by dividing the distance from the heart to the pulse wave detection site by the pulse wave transit time.

Here, the relationship between time T1 and time T2 does not have to be that at which the blood sent from the heart at time T1 actually reaches the pulse wave detection site at time T2. As described below, since the correlation between the pulse wave transit time (velocity) and the blood pressure value can be obtained from the actual measured values of these two parameters, as long as the definition of pulse wave transit time (velocity) is fixed, there are no problems when calculating the blood pressure value.

Further, in FIG. 4, to facilitate the description of the method for calculating the pulse wave transit time, the graph is depicted with the signal intensity of the electrocardiography waveform A having a greater value than the signal intensity of the pulse wave B. However, the relationship between the magnitude of the signal intensity of the electrocardiography waveform and the magnitude of the signal intensity of the pulse wave B is not limited to this example. Namely, as long as the positional relationship on the horizontal axis (time) between time T1 and time T2 is clear, the vertical axis (signal intensity) scale is not especially limited. The signal intensity of the electrocardiography waveform A and the signal intensity of the pulse wave B, for example, do not have to be plotted on the same vertical axis. Further, in order to accurately calculate the pulse wave transit time, the signal intensity of the electrocardiography waveform A and the signal intensity of the pulse wave B can be changed by appropriately designing the amplifier, for example, in the circuit illustrated in FIG. 3.

FIG. 5 is an expanded diagram illustrating a period close to time T1 and time T2 in FIG. 4 in an expanded manner. As illustrated in FIG. 5, when determining the pulse wave transit time, by providing a predetermined window (an upper limit and a lower limit) for time T2, the accuracy of the pulse wave transit time can be improved. Specifically, when time T2 is outside of the window illustrated in FIG. 5, for example, if time T2 is earlier than the edge on the time T1 side (time C) of the window, or if T2 is later than the edge on the opposite side of time T1 (time D) of the window, a pulse wave transit time calculated using that time T2 does not have to be employed. Here, the window width may be, for example, set based on an average value determined from past pulse wave transit time measurement data that was statistically acquired.

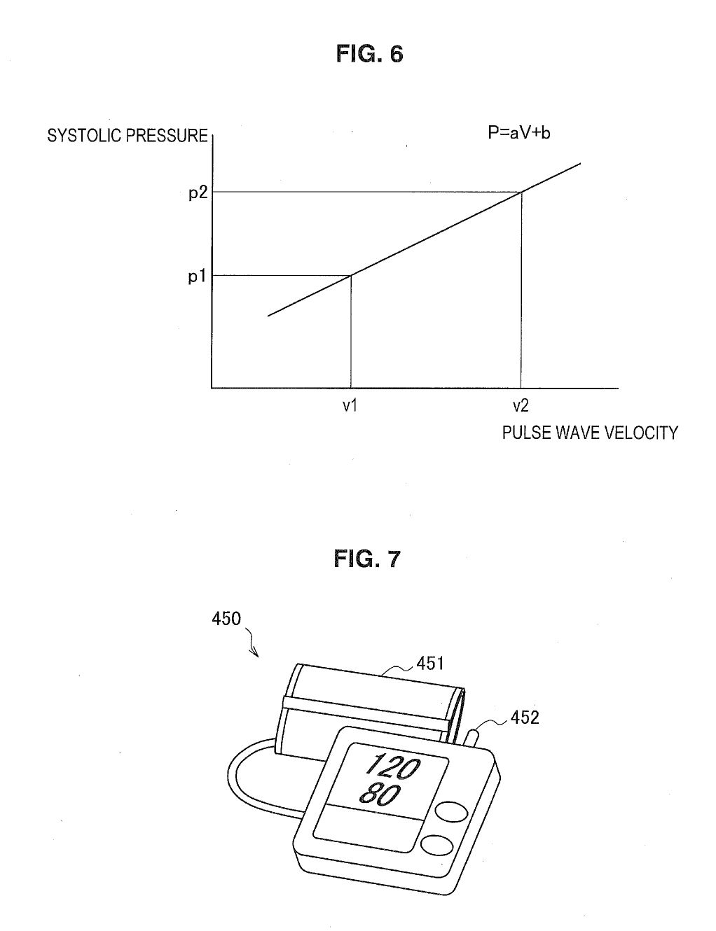

Next, the relationship between pulse wave velocity and the systolic pressure (maximum blood pressure) value will be described with reference to FIG. 6. FIG. 6 is a diagram illustrating a relationship between pulse wave velocity and a systolic pressure (maximum blood pressure) value. As illustrated in FIG. 6, there is a linear relationship P=aV+b (wherein P represents systolic pressure value, V represents pulse wave velocity, and a and b represent constants) between pulse wave velocity and the systolic pressure value. Therefore, if the values of constants a and b are known, the systolic pressure value can be determined based on the pulse wave velocity that is calculated from the measurement data. However, since there are individual differences in the above-described linear relationship, the values of constants a and b are determined according to the measurement subject.

To determine the values of constants a and b, it is only necessary to know two arbitrary points on the straight line P=aV+b. Therefore, for example, using a direct measurement method, the measurement subject measures a pulse wave velocity v1 and a systolic pressure p1 with respect to v1 when the measurement subject is in a given state (first state). Next, the measurement subject measures a pulse wave velocity v2 and a systolic pressure p2 with respect to v2 when the measurement subject is in a different state (second state). Constants a and b can then be determined using the values for the pulse wave velocity v1 and v2 and the systolic pressure p1 and p2. In the following description, the determination in this manner of the values of constants a and b, namely, the linear relationship between pulse wave velocity and the systolic pressure (maximum blood pressure) value, will be referred to as calibration. Here, the first state and the second state are not especially limited, as long as they are states that produce a certain level of difference or more in the systolic pressure value of the measurement subject. For example, the first state may be before exercise (at rest), and the second state may be immediately after exercise.

Further, in the above description, although a case was described in which the pulse wave velocity v1 and v2 and the systolic pressure p1 and p2 were measured at different states, if it can be assumed that there are no large individual differences for constant a, which represents the gradient of the straight line, calibration can also be carried out with only data from a single point for the pulse wave velocity v1 and the systolic pressure p1.

In the measurement apparatus 10 according to the first embodiment of the present disclosure, the blood pressure calculation unit 220 calculates the blood pressure value by utilizing the above-described linear relationship P=aV+b between pulse wave velocity and systolic pressure. Therefore, before starting blood pressure measurement, the measurement subject inputs the information for, for example, the pulse wave velocity v1 and v2 and the systolic pressure p1 and p2 in order to perform calibration. FIG. 7 illustrates an example of a blood pressure monitor for such calibration.

As illustrated in FIG. 7, a calibration blood pressure monitor 450 is, for example, a direct measurement blood pressure monitor. The measurement subject can measure blood pressure by, for example, wrapping a cuff 451 around his/her arm. Further, the calibration blood pressure monitor 450 may further include a communication unit 452. The calibration blood pressure monitor 450 can perform communication to and from the measurement apparatus 10 via the communication unit 452. Although the communication method between the calibration blood pressure monitor 450 and the measurement apparatus 10 may be wired or wireless, it is preferred that the communication unit 452 transmission method and the transmission method of the above-described communication unit in the measurement apparatus 10 are the same. For example, if the communication unit 452 communicates based on a wireless transmission method, then that transmission method is the same wireless transmission method as employed by the above-described communication unit in the measurement apparatus 10.

When the measurement subject's blood pressure is measured by the calibration blood pressure monitor 450, the calibration blood pressure monitor 450, for example, transmits the information relating to the measured systolic pressure to the measurement apparatus 10 via the communication unit 452. For example, in the example illustrated in FIG. 7, “120” is transmitted as a systolic pressure value from the calibration blood pressure monitor 450 to the measurement apparatus 10. On the other hand, simultaneously with the blood pressure measurement by the calibration blood pressure monitor 450, or preferably as close as possible to, the pulse wave velocity (time) of the measurement subject is also measured, and also transmitted to the measurement apparatus 10. Here, the pulse wave velocity (time) may be measured by a separate measurement apparatus, or may be measured by the chest contact measurement unit 100 of the measurement apparatus 10. Further, the calibration blood pressure monitor 450 may also be provided with a function for measuring the pulse wave velocity (time).

In addition, the systolic pressure and the pulse wave velocity (time) when the measurement subject is in different states are again measured as necessary, and the information relating to those measurements is transmitted to the measurement apparatus 10. The measurement apparatus 10 stores the information relating to the systolic pressure value and the information relating to the pulse wave velocity (time) in the storage unit 300, for example. The measurement apparatus 10 can perform calibration by using the information relating to the systolic pressure value and the information relating to the pulse wave velocity (time) stored in the storage unit 300.

Here, in the above description, although an example was described in which the pulse wave velocity v1 and v2 and the systolic pressure p1 and p2 were measured at different states when performing calibration, there may be three or more measurement points. Namely, calibration can be performed based on pulse wave velocity v1, v2, v3 . . . and systolic pressure p1, p2, p3 . . . at three or more different states. The greater the number of measurement states, the more accurate calibration is.

### 2.3. Appearance Example of the Measurement Apparatus

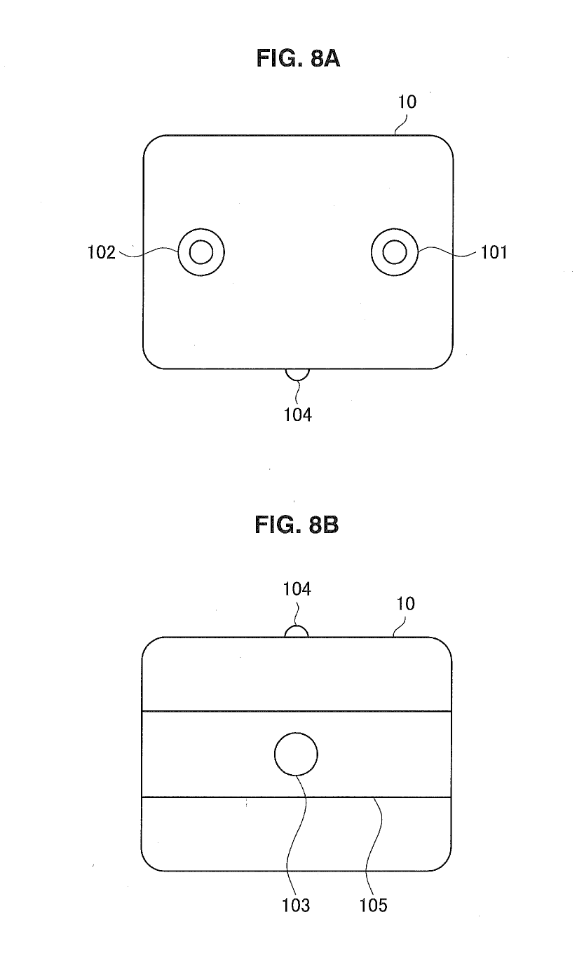

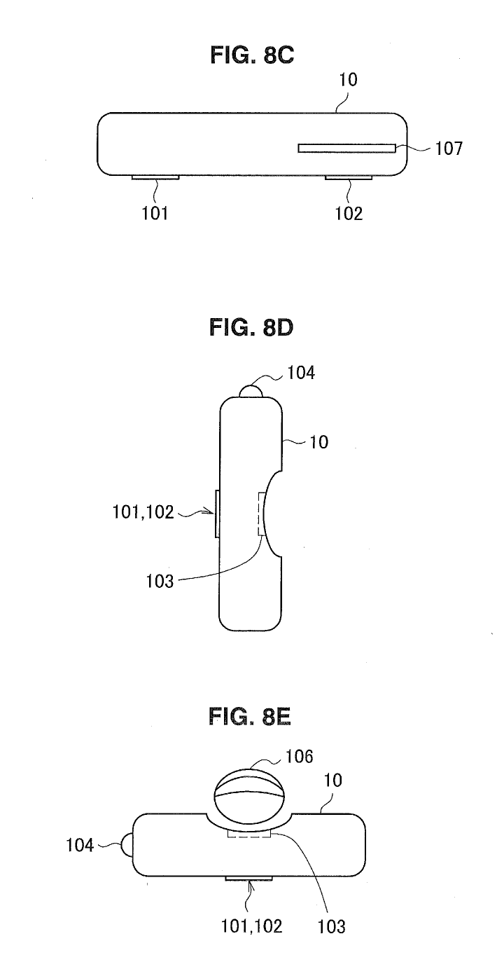

Next, an appearance example of the measurement apparatus 10 and a method for using the measurement apparatus 10 according to the first embodiment of the present disclosure will be described with reference to FIGS. 8 to 10.

First, an appearance example of the measurement apparatus 10 according to the first embodiment of the present disclosure will be described with reference to FIGS. 8A to 8E. FIG. 8A, FIG. 8B, FIG. 8C, and FIG. 8D are a rear view, a front view, a bottom view, and a side view, respectively, illustrating an appearance example of the measurement apparatus 10 according to this embodiment of the present disclosure. FIG. 8E is an explanatory diagram illustrating a positional relationship between the measurement apparatus 10 according to this embodiment of the present disclosure and a pulse wave measurement site when measuring a pulse wave.

As illustrated in FIGS. 8A to 8E, the measurement apparatus 10 according to the first embodiment of the present disclosure may have a roughly cuboid shape. As illustrated in FIG. 8A, electrode connector portions 101 and 102 are arranged on the rear face of the measurement apparatus 10 spaced apart from each other by a predetermined interval. Here, an electrode can be attached to each of the electrode connector portions 101 and 102. The electrodes attached to the electrode connector portions 101 and 102 correspond to the electrodes 111a and 111b of the electrocardiography measurement unit 110 illustrated in FIG. 3. Namely, the electrocardiography-related data of the measurement subject is measured by bringing the electrodes attached to the electrode connector portions 101 and 102 into contact with the chest of the measurement subject. Here, the interval between the electrode connector portion 101 and the electrode connector portion 102 may be appropriately designed in consideration of the electrocardiography measurement accuracy and the size of the measurement apparatus 10.

Further, the electrodes attached to the electrode connector portions 101 and 102 may be a dry electrode or a wet electrode. Namely, the electrodes attached to the electrode connector portions 101 and 102 may be switched between a dry electrode and a wet electrode based on the usage state of the measurement apparatus 10. In addition, the electrode connector portions 101 and 102 may be configured so that either a dry electrode or a wet electrode is constantly connected, and cannot be detached. If the electrode connector portions 101 and 102 are configured so that they cannot be detached from either of a dry electrode or a wet electrode, the electrode connector portions 101 and 102 and the dry electrode or wet electrode may be integrally formed. The configuration of the electrodes attached to the electrode connector portions 101 and 102 will be described below in more detail with reference to FIG. 9.

Still further, as illustrated in FIG. 8A, a LED 104 may be provided on one side face of the measurement apparatus 10. The LED 104 corresponds to the display unit 400 illustrated in FIG. 2. Here, the site where the LED 104 is provided is not limited to the site illustrated in FIG. 8A. This site may be appropriately changed, as long as it draws the measurement subject's attention.

Next, as illustrated in FIG. 8B, a pulse wave measurement detection window 103 is provided roughly in the center of the plane on the front face of the measurement apparatus 10, for example. Here, the pulse wave measurement detection window 103 corresponds to the optical sensing unit 121 of the pulse wave measurement unit 120 illustrated in FIG. 3. FIG. 8B illustrates a case in which the photodiode 123 detects reflected light from the pulse wave detection site, namely, when the LEDs 122a and 122b and the photodiode 123 are arranged on the same side as each other with respect to the pulse wave detection site. Therefore, the pulse wave-related data of the measurement subject is measured by bringing the pulse wave detection site into contact with the pulse wave measurement detection window 103.

Further, as illustrated in FIGS. 8B and 8D, a depression 105 may be formed in an area having a given width that includes the site where the pulse wave measurement detection window 103 is provided in the plane of the front face of the measurement apparatus 10. When measuring a pulse wave, as illustrated in FIG. 8E, the measurement subject can bring the pulse wave detection site, for example, a partial area of his/her finger, into contact with the pulse wave measurement detection window 103 while placing his/her finger so as to align with the depression 105.

Thus, in the measurement apparatus 10 according to this embodiment of the present disclosure, the electrocardiography measurement unit 110 and the pulse wave measurement unit 120 may be integrally formed. Here, the site where the pulse wave measurement detection window 103 is provided is not limited to the site illustrated in FIG. 8B. However, as illustrated in FIG. 8B, it is preferred to provide the pulse wave measurement detection window 103 on the face opposing the face on which the electrode connector portions 101 and 102, namely, the electrodes 111a and 111b of the electrocardiography measurement unit 110, are provided. Further, as illustrated in FIG. 8B, it is preferred to provide the pulse wave measurement detection window 103 at a position corresponding to between the electrode connector portion 101 and the electrode connector portion 102 in the plane opposing the face on which the electrode connector portions 101 and 102 are provided. This is because, as described in (2.1. Configuration of the measurement apparatus), when the electrocardiography measurement unit 110 and the pulse wave measurement unit 120 are in such a positional relationship, by bringing the pulse wave detection site of the measurement subject into contact with, or to press against, the pulse wave measurement unit 120, the electrocardiography measurement unit 110 contacts or presses against the chest measurement site of the measurement subject. Therefore, by providing the pulse wave measurement detection window 103 at the site illustrated in FIG. 8B, and by pressing the pulse wave detection site against the pulse wave measurement detection window 103, the electrode connector portions 101 and 102 can be made to reliably press against the chest measurement site.

Further, as illustrated in FIG. 8C, a slot 107 is provided on the bottom face of the measurement apparatus 10, for example. The slot 107 is a connection port for inserting a memory card, for example. Here, the site where the slot 107 is provided is not limited to the site illustrated in FIG. 8C. The slot 107 may be appropriately designed in consideration of ease of use for the measurement subject or by the user.

Next, a schematic configuration of the electrodes attached to the electrode connector portions 101 and 102 of the measurement apparatus 10 illustrated in FIGS. 8A to 8E will be described with reference to FIGS. 9A to 9D. FIG. 9A is a rear view (from the face that is brought into contact with the measurement subject's chest) illustrating an appearance example of a dry electrode according to the first embodiment of the present disclosure. FIG. 9B is a top view of the dry electrode illustrated in FIG. 9A. Further, FIG. 9C is a rear view (from the face that is brought into contact with the measurement subject's chest) illustrating an appearance example of a wet electrode according to the first embodiment of the present disclosure. FIG. 9D is a top view of the wet electrode illustrated in FIG. 9C.

As illustrated in FIGS. 9A and 9B, a dry electrode 501 has, for example, a protrusion 502 on a partial area of the front face thereof. By fitting the protrusion 502 to the electrode connector portion 101 or the electrode connector portion 102 illustrated in FIG. 8A, the dry electrode 501 is electrically connected to the measurement apparatus 10.

As illustrated in FIG. 9C, a wet electrode 503 has, for example, dry electrodes 504 and 505 on a partial area of the rear face thereof. The dry electrodes 504 and 505 are arranged spaced apart from each other by a predetermined interval, for example. Further, for example, conductive gels 506 and 507 are respectively coated around the periphery of the dry electrodes 504 and 505. The conductive gels 506 and 507 play the role of reducing the contact resistance between the dry electrodes 504 and 505 and the body when the wet electrode 503 is contacting the chest measurement site of the measurement subject. In addition, an adhesion portion 508 may be provided at an area on the rear face of the wet electrode 503 other than where the dry electrodes 504 and 505 and the conductive gels 506 and 507 are provided. By sticking the adhesion portion 508 to the chest measurement site of the measurement subject, the wet electrode 503 is fixed with the dry electrodes 504 and 505 and the conductive gels 506 and 507 in contact with the chest measurement site.

Moreover, as illustrated in FIG. 9D, the wet electrode 503 has, for example, protrusions 509 and 510 on a partial area on the front face thereof. The protrusions 509 and 510 are provided at a position corresponding to the dry electrodes 504 and 505 provided on the rear face side. By fitting the protrusions 509 and 510 to the electrode connector portions 101 and 102, respectively, illustrated in FIG. 8A, the wet electrode 503 is electrically connected to the measurement apparatus 10.

In the above, an example of the appearance of the measurement apparatus 10 according to the first embodiment of the present disclosure was described with reference to FIGS. 9A to 9D. Note that the appearance of the measurement apparatus 10 is not limited to the examples illustrated in FIGS. 8A to 8E and FIGS. 9A to 9D. For example, the measurement apparatus 10 may be some other shape than roughly cuboid. The shape of the front face and/or rear face of the measurement apparatus 10 may be various shapes, such as roughly egg shaped, roughly triangular shaped and the like. Further, for example, a display may be provided on a partial area of the front face of the measurement apparatus 10, and information about the measurement result and the like may be displayed on that display.

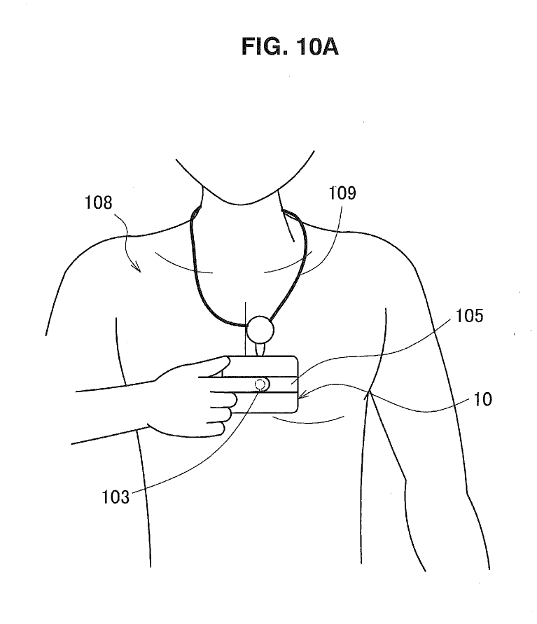

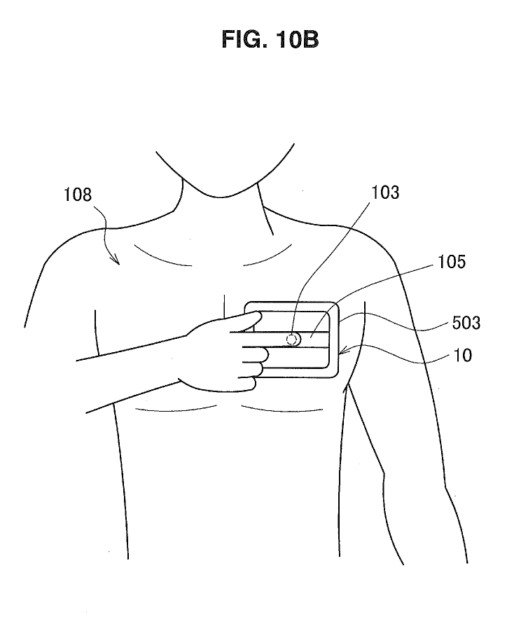

Next, an example of a method for using the measurement apparatus 10 according to the first embodiment of the present disclosure will be described with reference to FIGS. 10A and 10B. FIG. 10A is an explanatory diagram illustrating an example of a usage method when the measurement apparatus 10 according to the first embodiment of the present disclosure has a dry electrode. FIG. 10B is an explanatory diagram illustrating an example of a usage method when the measurement apparatus 10 according to the first embodiment of the present disclosure has a wet electrode.

As illustrated in FIG. 10A, when the measurement apparatus 10 according to the first embodiment of the present disclosure has a dry electrode, the measurement apparatus 10 may be made to hang down from the measurement subject's neck by a cord-like member 109 for hanging from the neck. Here, the length of the cord-like member 109 may be adjusted so that the measurement apparatus 10 is positioned at the height of the measurement subject's chest. Further, for example, the pulse wave measurement detection window 103 is provided at roughly the center of the front face of the measurement apparatus 10, and an electrode for electrocardiography measurement is provided on the rear face of the measurement apparatus 10. In this state, if the measurement subject presses the measurement apparatus 10 while touching the pulse wave measurement detection window 103 with the index finger on his/her right hand, for example, the electrode for electrocardiography measurement is pressed against the chest 108 of the measurement subject, whereby electrocardiography-related data and pulse wave-related data are simultaneously measured, and the measurement subject's blood pressure is calculated. Further, during measurement, by aligning the index finger on his/her right hand with the depression 105, the measurement subject can stabilize the contact with the pulse wave measurement detection window 103. Obviously, the measurement subject's blood pressure can be calculated even by pressing the measurement apparatus 10 against the chest with the left hand, and touching the pulse wave measurement detection window 103 with the index finger on the right hand.

Next, as illustrated in FIG. 10B, if the measurement apparatus 10 according to the first embodiment of the present disclosure has a wet electrode, the measurement apparatus 10 may be stuck to the chest measurement site of the measurement subject. Here, except for sticking the measurement apparatus 10 to the chest measurement site of the measurement subject with the adhesion portion 508 of the wet electrode 503 instead of hanging from the neck of the measurement subject with the cord-like member 109, the usage method illustrated in FIG. 10B is the same as the usage method illustrated in FIG. 10A. Accordingly, a detailed description thereof will be omitted. In a state in which the measurement apparatus 10 is stuck to the chest measurement site of the measurement subject, if the measurement subject presses the measurement apparatus 10 while touching the pulse wave measurement detection window 103 with the index finger on his/her right hand, for example, the electrode for electrocardiography measurement is pressed against the chest 108 of the measurement subject, whereby electrocardiography-related data and pulse wave-related data are simultaneously measured, and the measurement subject's blood pressure is calculated.

Further, if the measurement apparatus 10 is stuck to the chest measurement site of the measurement apparatus 10 with the adhesion portion 508 of the wet electrode 503, for example, the contact information acquisition unit 240 may acquire information indicating that the wet electrode 503 has separated from the chest measurement site. In addition, the measurement control unit 260 can also control the chest contact measurement unit 100 based on this information indicating that the wet electrode 503 has separated from the chest measurement site.

As described above, in the measurement apparatus 10 according to the first embodiment of the present disclosure, the electrocardiography measurement unit 110 and the pulse wave measurement unit 120 may be integrally formed. Further, when the measurement subject presses the measurement apparatus 10 against his/her chest while touching the pulse wave measurement detection window 103 with the index finger on his/her right hand, for example, electrocardiography-related data and pulse wave-related data are simultaneously measured, and the measurement subject's blood pressure is calculated. Therefore, since the electrocardiography-related data is measured in a state in which the electrocardiography measurement unit is reliably pressing against the chest measurement site, electrocardiography measurement can be accurately performed. Further, the measurement apparatus 10 can be carried around while hanging near to the chest measurement site of the measurement subject with a cord-like member, or while being stuck to the chest measurement site by a wet electrode for electrocardiography measurement. Consequently, the measurement subject can carry around the measurement apparatus 10 on a daily basis while the measurement apparatus 10 is basically in contact with the chest measurement site, which enables the measurement subject to casually perform blood pressure measurement.

### 3. Second Embodiment of the Present Disclosure

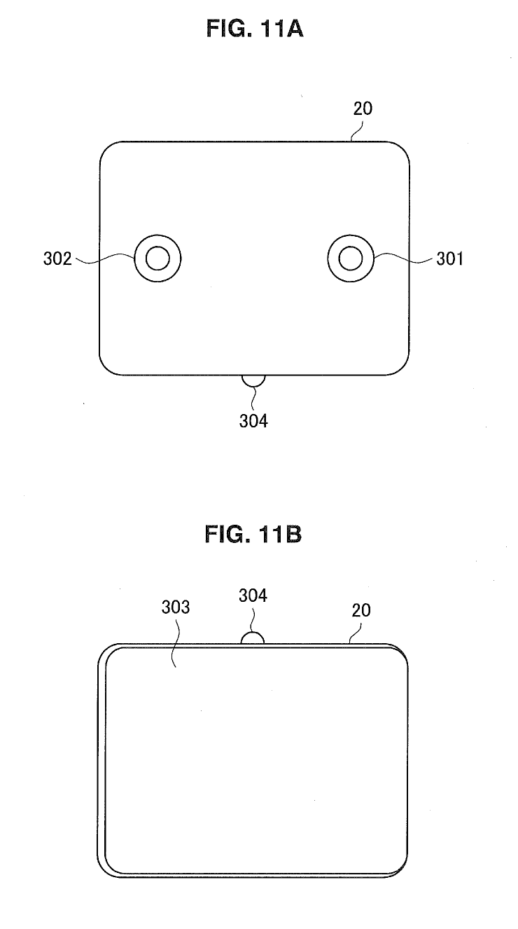

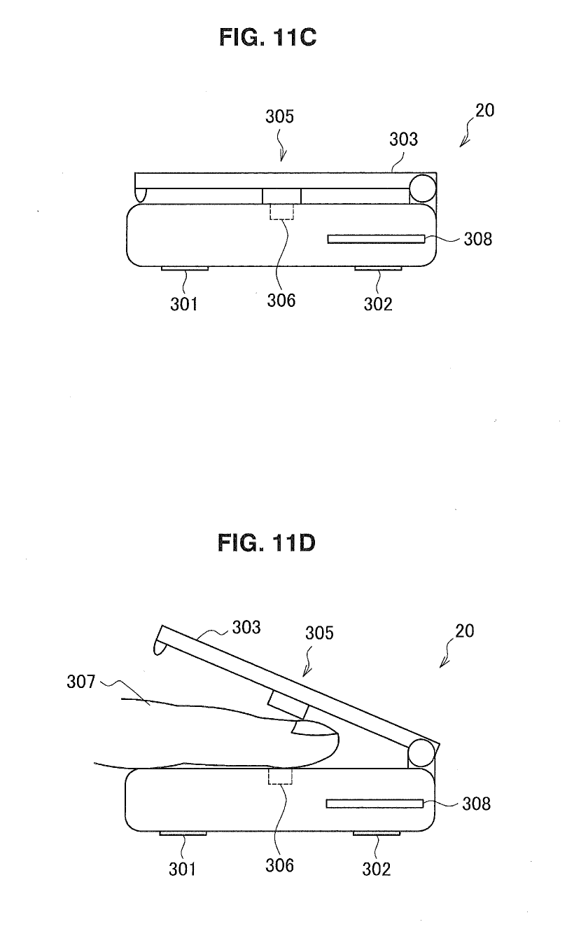

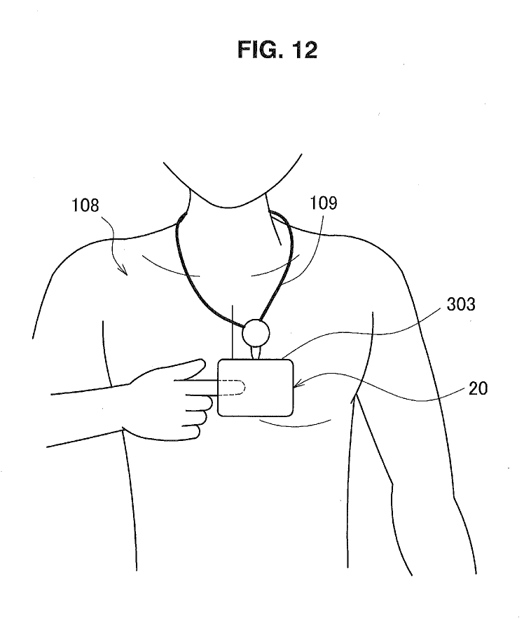

Next, an appearance example and a usage method of the measurement apparatus according to a second embodiment of the present disclosure will be described with reference to FIGS. 11 and 12. It is noted that since the measurement apparatus according to the second embodiment of the present disclosure has, except for the pulse wave measurement detection window, the same function and configuration as the measurement apparatus according to the first embodiment of the present disclosure, the following description will mainly be about this difference, and a detailed description of the other configurations will be omitted.

First, an appearance example of a measurement apparatus 20 according to the second embodiment of the present disclosure will be described with reference to FIG. 11. FIG. 11A, FIG. 11B, and FIG. 11C are a rear view, a front view, and a side view, respectively, illustrating an appearance example of the measurement apparatus 20 according to the second embodiment of the present disclosure. FIG. 11D is an explanatory diagram illustrating a positional relationship between the measurement apparatus 20 according to the second embodiment of the present disclosure and a pulse wave measurement site when measuring a pulse wave.

As illustrated in FIG. 11A, electrode connector portions 301 and 302 are arranged on the rear face of the measurement apparatus 20 according to the second embodiment of the present disclosure spaced apart from each other by a predetermined interval. Further, as illustrated in FIG. 11A, a LED 304 may also be provided on a side face of the measurement apparatus 20. Note that since the function and configuration of the electrode connector portions 301 and 302 and the LED 304 are the same as the electrode connector portions 101 and 102 and the LED 104, a detailed description thereof is omitted here.

As illustrated in FIG. 11B, a cover 303 is provided on the front face of the measurement apparatus 20 according to this embodiment of the present disclosure so as to cover the front face of the measurement apparatus 20. The cover 303 may be a plate-like member having roughly the same shape and surface area as the front face of the measurement apparatus 20. One side of the cover 303 is fixed to one side of the front face of the measurement apparatus 20 by a hinge. Namely, the cover 303 is configured so that it can be opened and closed with respect to the measurement apparatus 20 by pivoting on the side on which the hinge is provided. Here, a mechanism such as a spring or the like may be provided so that the cover 303 is normally in a closed state.

As illustrated in FIG. 11C, for example, a LED 305 is provided on the face of the cover 303 in contact with the measurement apparatus 20. The LED 305 plays the role of irradiating light on the pulse wave detection site. Here, the LED 305 corresponds to at least either the LED 122a or LED 122b. Namely, the LED 305 may be a single LED, or may be configured from two LEDs arranged in parallel. On the other hand, a pulse wave measurement detection window 306, for example, is provided in a partial area of the face covered by the cover 303 of the measurement apparatus 20. Here, the pulse wave measurement detection window 306 corresponds to the photodiode 123 illustrated in FIG. 3.

When measuring a pulse wave, as illustrated in FIG. 11D, the pulse wave detection site of the measurement subject is sandwiched between the cover 303 and the face covered by the cover 303 of the measurement apparatus 20. More specifically, the pulse wave detection site of the measurement subject is sandwiched between the LED 305 provided on the cover 303 and the pulse wave measurement detection window 306. Namely, the pulse wave detection site of the measurement subject is sandwiched between a light irradiation unit and a light incident portion provided on the cover 303. Therefore, among the light irradiated by the LED 305, the pulse wave measurement detection window 306 can detect light that has passed through the pulse wave detection site of the measurement subject.

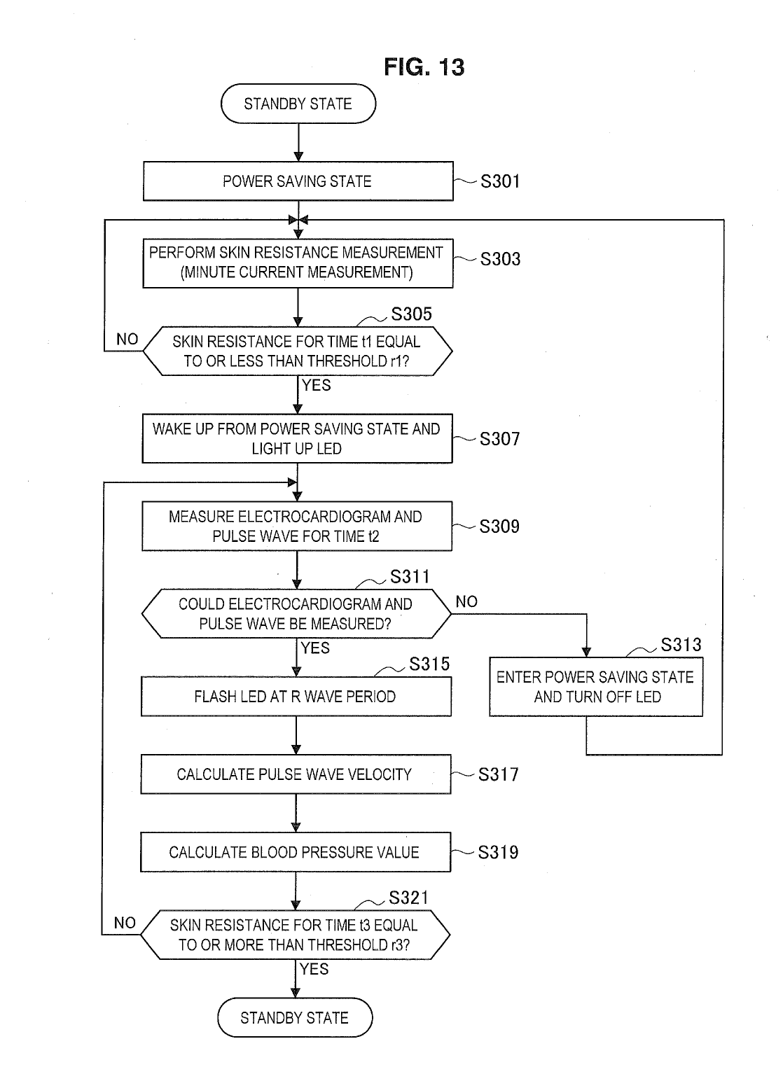

Next, a method for using the measurement apparatus 20 according to the second embodiment of the present disclosure will be described with reference to FIG. 12. FIG. 12 is an explanatory diagram illustrating an example of a usage method when the measurement apparatus 20 according to the second embodiment of the present disclosure has a dry electrode. It is noted that, other than the pulse wave measurement method, since the usage method example illustrated in FIG. 13 is the same as the usage method example illustrated in FIG. 10, the following description will mainly only be about this difference.

As illustrated in FIG. 12, when the measurement apparatus 20 according to this embodiment of the present disclosure has a dry electrode, the measurement apparatus 20 may be made to hang down from the measurement subject's neck by a cord-like member 109 for hanging from the neck. Further, the length of the cord-like member 109 may be adjusted so that the measurement apparatus 20 is positioned at the height of the measurement subject's chest. In this state, the measurement subject presses the measurement apparatus 20 in the direction of the chest 108 while sandwiching the pulse wave detection site, for example, a finger, between the face covered by the cover 303 of the measurement apparatus 20 and the cover 303. Since an electrode for electrocardiography measurement is provided on the rear face of the measurement apparatus 20, by pressing the measurement apparatus 20 while the measurement subject's finger is sandwiched, the electrode for electrocardiography measurement is pressed against the chest 108 of the measurement subject, whereby electrocardiography-related data and pulse wave-related data are simultaneously measured, and the measurement subject's blood pressure is calculated.

Thus, the measurement apparatus 20 according to the second embodiment of the present disclosure includes a cover 303 provided so as to cover the front face of the measurement apparatus 20. Further, by sandwiching the pulse wave detection site between the face covered by the cover 303 of the measurement apparatus 20, a pulse wave can be measured. By having such a configuration, the position of the pulse wave detection site is fixed with respect to the measurement apparatus 20, so that pulse wave measurement can be performed more accurately.

Here, the embodiment described for the measurement apparatus 10 according to according to the first embodiment of the present disclosure can be applied to a large extent for the measurement apparatus 20 according to the second embodiment of the present disclosure. For example, the measurement apparatus 20 according to the second embodiment of the present disclosure may include a wet electrode. If the measurement apparatus 20 according to the second embodiment of the present disclosure does include a wet electrode, as illustrated in FIG. 10B, the measurement apparatus 20 can be stuck to the chest measurement site of the measurement subject.

### 4. Calculation Method Processing Procedure

Next, the calculation method processing procedure according to the first embodiment and the second embodiment of the present disclosure will be described with reference to FIG. 13. FIG. 13 is a flow diagram illustrating a blood pressure measurement processing procedure for the calculation method according to the first and second embodiments of the present disclosure. It is noted that in the following description, before step S301, a calibration operation has been performed by the measurement subject, and information relating to the linear relationship (P=aV+b) between pulse wave velocity and the systolic pressure value has been input into the measurement apparatus 10 or 20.

As illustrated in FIG. 13, first, the measurement apparatus 10 or 20 is in a power standby state (power saving state) (step S301). In a power standby state, when the skin resistance detection device 112 of the electrocardiography measurement unit 110 detects a resistance value between electrode 111a and electrode 111b based on the detection of a minute current between the two electrodes (step S303), the measurement control unit 260 of the control unit 200 determines whether that resistance value is equal to or less than a predetermined threshold r1 for a predetermined time t1 (step S305). Here, a state in which a resistance value between electrode 111a and electrode 111b is detected based on the detection of a minute current between the two electrodes is realized by, for example, the electrodes 111a and 111b contacting the skin of the measurement subject. Further, the contact site when the electrodes 111a and 111b contact the skin of the measurement subject may be the chest measurement site for performing electrocardiography measurement.

In step S305, if it is determined that the resistance value between the electrodes 111a and 111b is not equal to or less than the predetermined threshold r1 for the predetermined time t1, the measurement control unit 260 determines that the electrodes 111a and 111b and the chest measurement site are not in sufficient contact, and the processing returns to step S303. On the other hand, in step S305, if it is determined that the resistance value between the electrodes 111a and 111b is equal to or less than the predetermined threshold r1 for the predetermined time t1, the measurement control unit 260 determines that the electrodes 111a and 111b and the chest measurement site are in contact, and the electrocardiography measurement unit 110 performs a control to start electrocardiography measurement (step S307). Further, the measurement apparatus 10 or 20 wakes up from the power standby state. Here, in step S307, to indicate that the power standby state has ended and that electrocardiography measurement has been started, the display control unit 230 can control the display unit 400 so that the LED 104 or 304 light up, for example. Further, the fact that the power standby state has ended may also be transmitted some other way, such as by a buzzer or a vibrator, for example. Here, the control to wake up the measurement apparatus 10 or the measurement apparatus 20 from the power standby state is performed by the control unit 200, for example.

Following step S307, electrocardiography measurement and pulse wave measurement are performed for a time t2 (step S309). Next, in step S311, a determination is made regarding the reliability of the measured electrocardiography-related data and pulse wave-related data. Specifically, the measurement state determination unit 250 of the control unit 200 determines whether the electrocardiography-related data was appropriately measured based on information (contact information) relating to the contact state between the electrodes 111a and 111b and the chest measurement site. More specifically, the measurement state determination unit 250 may determine whether the electrocardiography-related data of the measurement subject was appropriately measured based on, for example, whether a resistance value that is included in the contact information between the electrode 111a and the electrode 111b is smaller than a pre-set threshold (first threshold). Here, the resistance value between the electrode 111a and the electrode 111b changes according to the strength that the electrocardiography measurement unit 110 is pressing on the chest measurement site of the measurement subject. Therefore, the determination regarding the reliability of the measurement performed by the electrocardiography measurement unit 110 can be performed based on the resistance value between the electrode 111a and the electrode 111b.

Further, as described with reference to FIGS. 10A and 10B and FIG. 12, the measurement subject can also bring the measurement apparatus 10 or 20 into contact with his/her chest, for example, in a state in which the pulse wave detection site has been brought into contact with the pulse wave measurement detection window 103 or 306. Therefore, the contact information can indirectly include information relating to the contact state (contact presence/absence, pressing strength etc.) between the pulse wave detection site and the pulse wave measurement unit 120. Therefore, in step S311, the measurement state determination unit 250 can determine whether the pulse wave-related data was appropriately measured based on contact information. Specifically, the measurement state determination unit 250 may determine whether the pulse wave-related data of the measurement subject was appropriately measured based on, for example, whether a resistance value that is included in the contact information between the electrode 111a and the electrode 111b is smaller than a pre-set threshold (second threshold).

It is noted that the first threshold and the second threshold may be the same value or a different value to each other. Further, the first threshold and the second threshold may be, for example, the r1 in step S305, or some other value. Although not especially limited, the first threshold and the second threshold can be appropriately set based on past measurement data or the like that has been statistically acquired. Further, the first threshold and the second threshold may be freely set by the measurement subject before starting measurement.

Further, in step S311, in addition to contact information, the measurement state determination unit 250 may also determine the reliability of the measured electrocardiography-related data and/or the pulse wave-related data of the measurement subject by comparing with previously measured data. For example, if the measured electrocardiography-related data and/or the pulse wave-related data of the measurement subject is greatly different from the previously measured data, the measurement state determination unit 250 can determine that measurement was not performed appropriately.

In step S311, if it is determined that the electrocardiography-related data and/or the pulse wave-related data of the measurement subject was not appropriately measured, the electrocardiography measurement and/or pulse wave measurement is/are finished by the measurement control unit 260. The measurement apparatus 10 or 20 proceeds to a power standby state, and if the LED 104 or 304 were lit up in step S307, the LED 104 or 304 may be turned off (step S313). The processing then returns to step S303, and the measurement apparatus 10 or 20 waits until contact between the electrodes 111a and 111b and the skin of the measurement subject is detected.

In step S311, if it is determined that the electrocardiography-related data and/or the pulse wave-related data of the measurement subject was appropriately measured, the fact that the measurement was performed appropriately is notified to the measurement subject. For example, the display control unit 230 may flash the LED 104 or 304 in synchronization with the period of the R wave of the electrocardiography waveform of the measurement subject (step S315). Further, if the display unit 400 has a display, the measured electrocardiography-related data, pulse wave-related data and/or pulse wave-related data and the like can be displayed on that display in numerical or graphical form, for example.

Next, in step S317, based on the measured electrocardiography-related data and pulse wave-related data, the blood pressure calculation unit 220 calculates the pulse wave velocity (time) of the measurement subject. Further, in step S319, based on the calculated pulse wave velocity (time), the blood pressure calculation unit 220 calculates the measurement subject's blood pressure. Since the pulse wave velocity (time) and the blood pressure calculation method were described in (2.2. Blood pressure calculation method), a detailed description thereof is omitted here. Here, the blood pressure value calculated in step S319 may also be displayed on the display or the like of the display unit 400 under the control of the display control unit 230. Further, the blood pressure value calculated in step S319 may be stored in the storage unit 300 and/or an external storage unit. In addition, the blood pressure value calculated in step S319 may be transmitted to an external device via a communication unit.

After the blood pressure value has been calculated in step S319, in step S321, based on the contact information, the measurement control unit 260 determines whether the resistance value between the electrode 111a and the electrode 111b is equal to or more than a predetermined threshold r3 for a predetermined time t3 (step S321). If it is determined that the resistance value between the electrode 111a and the electrode 111b is equal to or more than the predetermined threshold r3 for the predetermined time t3, the measurement control unit 260 determines that the electrode 111a and the electrode 111b have been separated from the chest measurement site of the measurement subject, and finishes the measurement performed by the chest contact measurement unit 100. When measurement has finished, if the measurement apparatus 10 or 20 enters a power standby state and the LED 104 or 304 is flashing, the measurement control unit 260 can also turn off the LED 104 or 304. Here, the control for making the measurement apparatus 10 or 20 enter a power standby state and the control for making the measurement apparatus 10 enter a power standby state are performed by the control unit 200, for example.

In step S321, if it is determined that the resistance value between the electrode 111a and the electrode 111b is not equal to or more than the predetermined threshold r3 for the predetermined time t3, the measurement control unit 260 determines that the electrode 111a and the electrode 111b are still in contact with the chest measurement site. Then, the processing returns to step S309, and blood pressure is again measured by performing measurement with the chest contact measurement unit 100.

In the above, although the calculation method processing procedure according to the first and second embodiments of the present disclosure was described with reference to FIG. 13, the calculation method processing procedure according to the first and second embodiments of the present disclosure is not limited to this example. For example, in the example illustrated in FIG. 13, in steps S309 and S11, although the electrocardiography-related data and the pulse wave-related data of the measurement subject were simultaneously measured, and the reliability of each measurement data was determined simultaneously, these pieces of measurement data may be measured separately, and their reliability may be determined separately. If the electrocardiography-related data and the pulse wave-related data are measured separately, an example of the blood pressure measurement processing procedure is as follows. For example, first, in a state in which the measurement subject has brought the electrocardiography measurement unit 110 of the measurement apparatus 10 or 20 into contact with his/her chest, and the pulse wave detection site is not in contact with the pulse wave measurement unit 120, only the electrocardiography measurement is performed, and whether the electrocardiography-related data has been appropriately measured is confirmed. Next, after it has been determined that the electrocardiography measurement was performed appropriately, the measurement subject brings the pulse wave detection site, for example his/her finger, into contact with the pulse wave measurement unit 120, and pulse wave measurement can be performed. Thus, by performing the electrocardiography measurement and pulse wave measurement in order, if the electrocardiography measurement and/or the pulse wave measurement were not performed appropriately, it is clear for which measurement there is a problem. Here, even if the electrocardiography measurement and the pulse wave measurement are performed in order, it is still preferred that the electrocardiography-related data and the pulse wave-related data to be used in calculating the blood pressure are data that were measured almost simultaneously.

Further, the times t1, t2, and t3, and the thresholds r1 and r3 may be, for example, appropriately set based on past measurement data or the like that was statistically acquired. For example, t1 may be 2 (seconds), t2 may be 3 (seconds), and t3 may be 10 (seconds). Further, thresholds r1 and r3 may be the same or different values from each other. In addition, thresholds r1 and r3 may be the same value as the first threshold or the second threshold, for example.

### 5. Modified Examples of the First and Second Embodiments of the Present Disclosure

### 5.1. Modified Example of the Chest Contact Measurement Unit

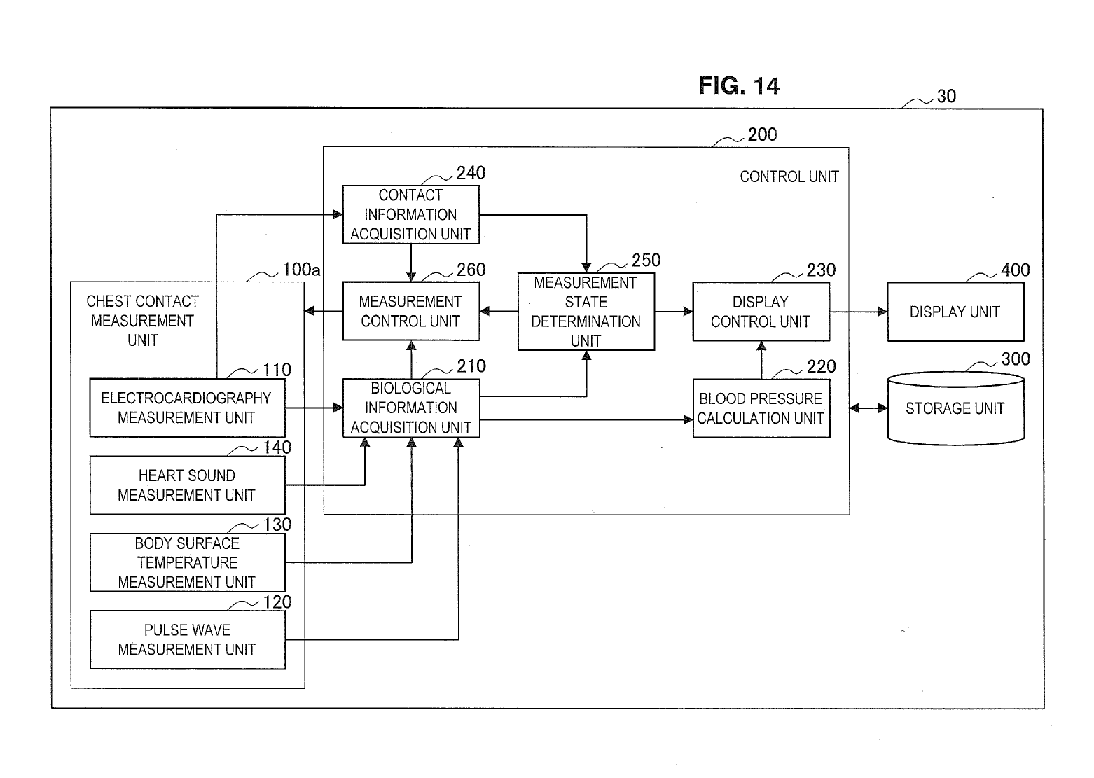

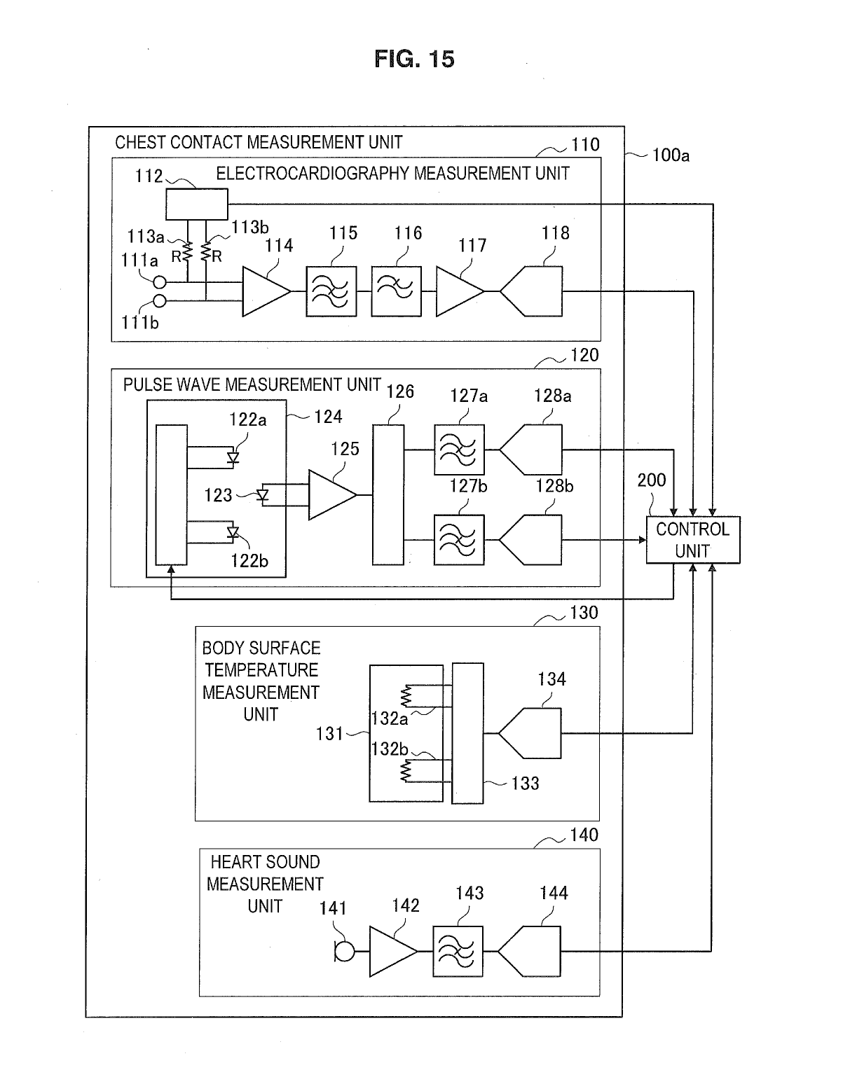

Next, modified examples of the first and second embodiments of the present disclosure will be described. First, a different configuration example of the chest contact measurement unit will be described with reference to FIGS. 14 and 15.

FIG. 14 is a function block diagram illustrating an example of a different configuration of the measurement apparatus according to the first and second embodiments of the present disclosure. FIG. 15 is a function block diagram illustrating a schematic configuration of a chest contact measurement unit of the measurement apparatus illustrated in FIG. 14.

As illustrated in FIG. 14, a measurement apparatus 30 according to this embodiment of the present disclosure includes, for example, a chest contact measurement unit 100a, a control unit 200, a storage unit 300, and a display unit 400. It is noted that, other than the function and configuration of the chest contact measurement unit 100a, since the measurement apparatus 30 illustrated in FIG. 14 is the same as the measurement apparatus 10 illustrated in FIG. 2, the following description will mainly only be about this difference, and a detailed description of the other configurations will be omitted.

As illustrated in FIG. 15, the chest contact measurement unit 100a according to this embodiment of the present disclosure includes an electrocardiography measurement unit 110, a pulse wave measurement unit 120, a body surface temperature measurement unit 130, and a heart sound measurement unit 140. It is noted that since the function and configuration of the electrocardiography measurement unit 110 and the pulse wave measurement unit 120 are the same as the electrocardiography measurement unit 110 and pulse wave measurement unit 120 illustrated in FIGS. 2 and 3, a description thereof will be omitted. Therefore, in the following, a schematic configuration of the body surface temperature measurement unit 130 and the heart sound measurement unit 140 will be described with reference to FIG. 15.

As illustrated in FIG. 15, the body surface temperature measurement unit 130 is configured from, for example, a thermopile sensor 131, a temperature signal generation unit 133, and an AD convertor 134.

The thermopile sensor 131 is a thermal infrared sensor that detects infrared rays radiated from a target object, and generates a thermoelectromotive force based on the amount of incident energy of those infrared rays. Here, the thermopile sensor 131 is configured from, for example, an infrared sensor 132a and a reference temperature sensor 132b. The infrared sensor 132a is a sensor for detecting infrared rays radiated from the target object. The reference temperature sensor 132b is a sensor for measuring ambient temperature. The thermopile sensor 131, for example, inputs a difference between a measured output signal from the infrared sensor 132a and an output signal from the reference temperature sensor 132b to the temperature signal generation unit 133.

The temperature signal generation unit 133 can calculate the temperature of the target object based on the output signal from the infrared sensor 132a and the output signal from the reference temperature sensor 132b. Further, in addition to a calculation circuit for calculating the temperature of the target object, the temperature signal generation unit 133 may also include another circuit such as an amplification circuit and the like. The temperature signal generation unit 133 inputs a signal relating to the calculated temperature of the target object into the AD convertor 134.

The AD convertor 134 converts the analog signal input from the temperature signal generation unit 133 into a digital signal, and transmits the converted digital signal to the biological information acquisition unit 210 of the control unit 200. The information relating to the temperature of the target object transmitted to the biological information acquisition unit 210 may be stored in the storage unit 300 or in an external storage unit, or even transmitted to an external device via a communication unit.

Further, as illustrated in FIG. 15, the heart sound measurement unit 140 is configured from, for example, a microphone 141, a microamp 142, a band-pass filter 143, and an AD converter 144.

The microphone 141 is, for example, a capacitor-type microphone 141 that inputs a signal relating to heart sound into the microamp 142.

The microamp 142 amplifies the input signal relating to heart sound, and inputs the amplified signal into the band-pass filter 143. The band-pass filter 143 removes frequency components other then the desired frequency component from the input signal relating to heart sound, and inputs the resultant signal into the AD converter 144. Here, the microamp 142 gain, the band-pass filter 143 cutoff band and the like may be appropriately set in consideration of the accuracy of the measurement data relating to heart sound, the subsequent signal processing methods and the like.

The AD converter 144 converts the analog signal relating to heart sound input from the band-pass filter 143 into a digital signal, and transmits the converted digital signal to the biological information acquisition unit 210 of the control unit 200. The information relating to heart sound transmitted to the biological information acquisition unit 210 may be stored in the storage unit 300 or in an external storage unit, or even transmitted to an external device via a communication unit.

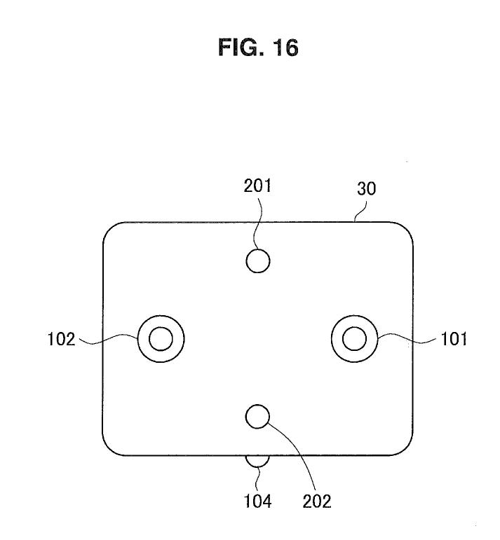

Further, as illustrated in FIG. 16, the detection units for the body surface temperature measurement unit 130 and the heart sound measurement unit 140 may be provided on the face of the measurement apparatus 30 on which the electrode for electrocardiography measurement is arranged, for example. FIG. 16 is a rear view illustrating an appearance example of the measurement apparatus 30 illustrated in FIG. 14.

As illustrated in FIG. 16, on the rear face of the measurement apparatus 30 according to this embodiment of the present disclosure, in addition to the electrode connector portions 101 and 102 for electrocardiography measurement, for example, a body surface temperature measurement detection window 201 and a heart sound measurement detection window 202 are provided. The body surface temperature measurement detection window 201 corresponds to the thermopile sensor 131 illustrated in FIG. 15. The heart sound measurement detection window 202 corresponds to the microphone 141 illustrated in FIG. 15. Therefore, in order to measure the data relating to the heart sound of the measurement subject, when the electrode on the rear face of the measurement apparatus 30 is brought into contact with the chest measurement site of the measurement subject, the body surface temperature measurement detection window 201 and the heart sound measurement detection window 202 are also brought into contact with the chest of the measurement subject. Namely, when measuring the data relating to heart sound, the data relating to the body surface temperature and the data relating to the heart sound of the measurement subject can be simultaneously measured. Further, although not illustrated, if the electrode for electrocardiography measurement is a wet electrode, the measurement apparatus 30 may be configured so that the adhesion portion 508 illustrated in FIG. 9C is not provided at a position corresponding to the body surface temperature measurement detection window 201 and the heart sound measurement detection window 202.

As described above, in the measurement apparatus 30, which is a different configuration example from the measurement apparatuses 10 and 20 according to the first and second embodiments of the present disclosure, the chest contact measurement unit 100a includes the electrocardiography measurement unit 110, the pulse wave measurement unit 120, the body surface temperature measurement unit 130, and the heart sound measurement unit 140. Further, in addition to electrocardiography measurement and pulse wave measurement, data relating to the body surface temperature (body surface temperature measurement information) and data relating to the heart sound (heart sound information) of the measurement subject can also be measured. These pieces of information, i.e., the body surface temperature measurement information and the heart sound information, may be used when performing calibration, for example. By utilizing the body surface temperature measurement information and the heart sound information when performing calibration, the linear relationship (P=aV+b) between pulse wave velocity and systolic pressure can be determined more accurately. Here, the electrocardiography measurement unit 110, the pulse wave measurement unit 120, the body surface temperature measurement unit 130, and the heart sound measurement unit 140 may be integrally configured.

In the above, an example of the functions of the measurement apparatus 30 according to this embodiment of the present disclosure, especially an example of the function of the chest contact measurement unit 100a, was described in detail with reference to FIGS. 14 and 15. Further, an appearance example of the measurement apparatus 30 illustrated in FIG. 14 was described with reference to FIG. 16. Here, the respective constituent elements illustrated in FIGS. 14 and 15 may be configured using versatile parts and circuits, or may be configured from hardware that is specialized to the function of each constituent element.

Further, although the circuit configuration of the body surface temperature measurement unit 130 and the heart sound measurement unit 140 were described in detail with reference to FIG. 15, these circuit configurations are not limited to the illustrated example. The circuit configuration of the body surface temperature measurement unit 130 and the heart sound measurement unit 140 can be appropriately changed as long as the above-described desired functions can be realized.

### 5.2. Modified Example of the Configuration of the Measurement Apparatus

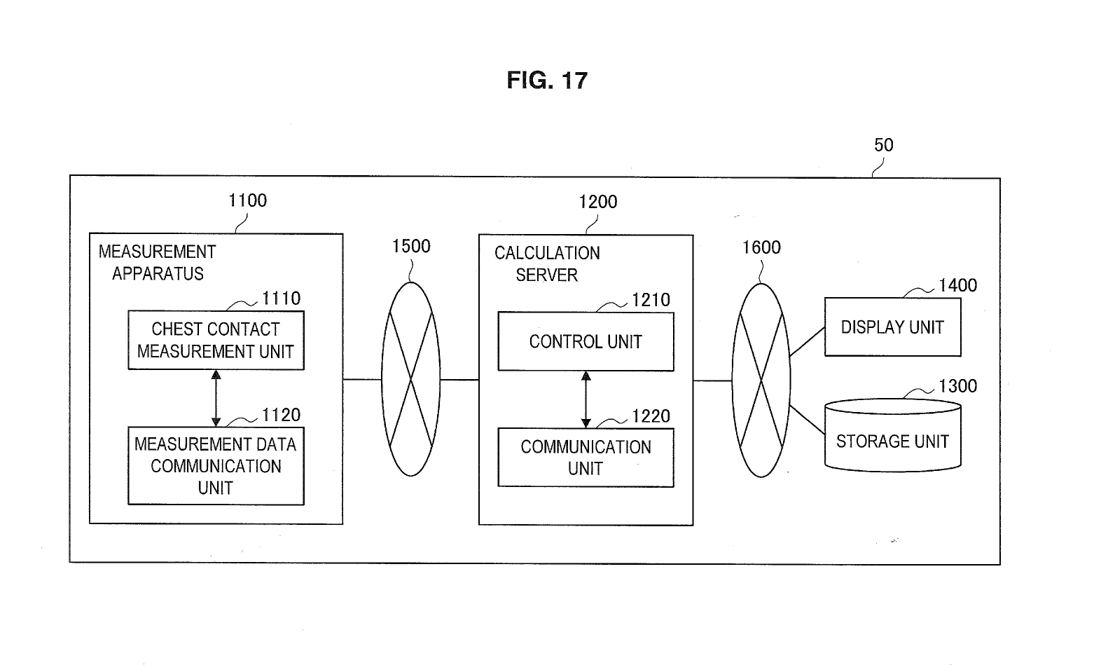

Next, a modified example of the configuration of the measurement apparatus according to the first and second embodiments of the present disclosure will be described. In the above description, although cases were described in which the measurement apparatus according to the first and second embodiments of the present disclosure was an integrated apparatus, the present technology is not limited to this. The measurement apparatus according to the first and second embodiments of the present disclosure may also be configured from a plurality of apparatuses based on the functions of the measurement apparatus. A system example in which the measurement apparatus according to the first and second embodiments of the present disclosure is configured from a plurality of apparatuses will now be described with reference to FIG. 17.

FIG. 17 is a function block diagram illustrating a schematic configuration of a measurement system 50 according to the first and second embodiments of the present disclosure. As illustrated in FIG. 17, the measurement system 50 according to the first and second embodiments of the present disclosure is configured from, for example, a measurement apparatus 1100, a calculation server 1200, a storage unit 1300, a display unit 1400, and networks 1500 and 1600. Here, the measurement apparatus 1100 and the calculation server 1200 may be configured so that they can communicate with each other via the network 1500. The calculation server 1200, the storage unit 1300, and the display unit 1400 may be configured so that they can communicate with each other via the network 1600. Further, since the function and the configuration of the storage unit 1300 and the display unit 1400 are the same as that of the storage unit 300 and display unit 400 illustrated in FIGS. 2 and 14, a detailed description thereof is omitted here.

The measurement apparatus 1100 has, for example, a chest contact measurement unit 1100 and a measurement data communication unit 1120. Here, since the function and the configuration of the chest contact measurement unit 1100 are the same as that of the chest contact measurement unit 100 or the chest contact measurement unit 100a illustrated in FIGS. 2, 3, 14, and 15, a detailed description thereof is omitted here.

The measurement data communication unit 1120 transmits the various types of data measured by the chest contact measurement unit 1110 to a below-described communication unit 1220 of the calculation server 1200 via the network 1500. Here, the various types of data transmitted to the measurement data communication unit 1120 may be, for example, electrocardiography information, pulse wave information, contact information, body surface temperature information, and/or heart sound information and the like.

The calculation server 1200 has, for example, a control unit 1210 and a communication unit 1220. The communication unit 1220 can perform communication to and from the measurement apparatus 1100 via the network 1500. Further, the communication unit 1220 can perform communication to and from the storage unit 1300 and the display unit 1400 via the network 1600. The communication unit 1220, for example, receives the various types of data transmitted from the measurement data communication unit 1120, and transmits the received data to the control unit 1210.

The control unit 1210 performs processing such as calculating the measurement subject's blood pressure based on the various types of received data. Further, the control unit 1210 controls the measurements performed by the measurement apparatus 1100. Since the function and configuration of the control unit 1210 are the same as the control unit 200 illustrated in FIGS. 2 and 14, a detailed description thereof is omitted here. The control unit 1210 transmits the processed results, various control commands and the like to the communication unit 1220.

The communication unit 1220 transmits the results processed by the control unit 1210, the various control commands and the like to, for example, the measurement data communication unit 1120, the storage unit 1300 and/or the display unit 1400 of the measurement apparatus 1100.

In the above, the measurement system 50 according to the first and second embodiments of the present disclosure was described with reference to FIG. 17. As illustrated in FIG. 17, by configuring the measurement system from a plurality of the measurement apparatuses according to the first and second embodiments of the present disclosure, the following advantageous effects can be obtained.

For example, if the measurement subject wants to measure his/her blood pressure when away from his/her home or office, the measurement subject only has to carry the measurement apparatus 1100. Other units, such as the calculation server 1200, the storage unit 1300, and the display unit 1400 can be installed in a server center or the measurement subject's home. Since it is sufficient for the measurement apparatus 1100 to have a configuration that measures data relating to biological activity, compared with a case in which all of the functions are integrated, the apparatus can be made more compact and lighter, and thus have superior portability.

Further, for example, by installing the display unit 1400 at a remote family residence of the measurement subject, the measurement subject's family can know the measurement results of the measurement subject while being at a remote location. Further, if there are a plurality of display units 1400, one of the display units 1400 can be installed at a location where the measurement subject can immediately confirm the results, and another display unit 1400 can be installed at the remote family residence.

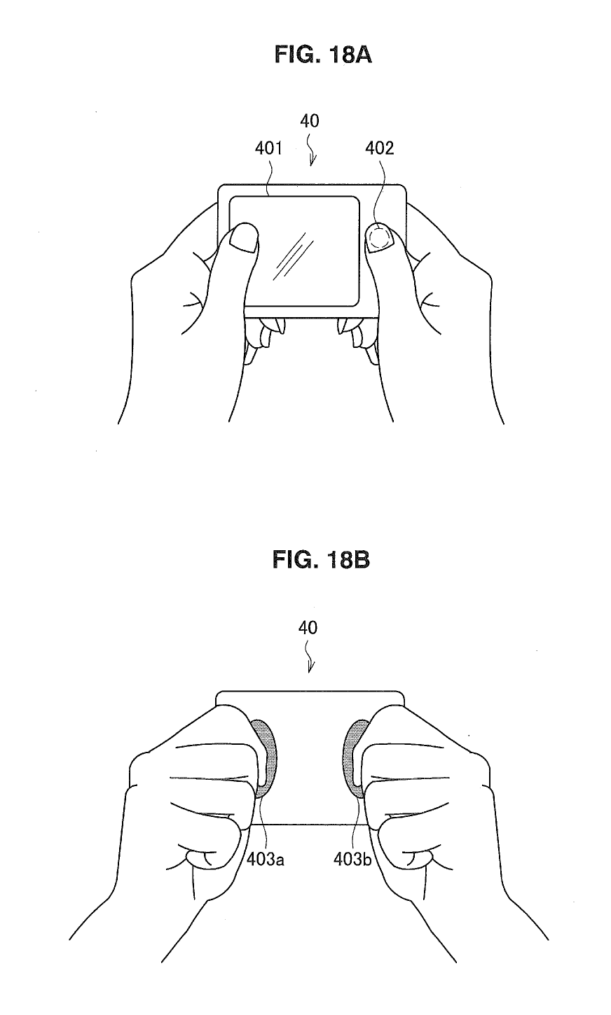

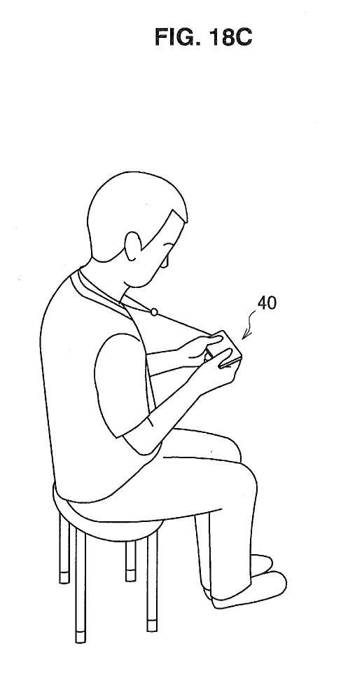

Next, a modified example of the usage method of the measurement apparatus according to the first and second embodiments of the present disclosure will be described. In the above description, although cases were described in which blood pressure was measured by bringing the measurement apparatus according to the first and second embodiments of the present disclosure into contact with the chest, the present technology is not limited to this. For example, blood pressure can also be measured by the measurement subject holding the measurement apparatus in both hands. A method in which blood pressure is measured by the measurement subject holding the measurement apparatus in both hands will now be described with reference to FIGS. 18A to 18C.

FIG. 18A is a schematic diagram illustrating how a measurement apparatus 40 according to the first and second embodiments of the present disclosure looks when held in both hands by the measurement subject. FIG. 18B is an expanded view illustrating how the hands of the measurement subject in FIG. 18A look from the measurement subject side. FIG. 18C is an expanded view illustrating how the hands of the measurement subject in FIG. 18A look from the reverse side of the measurement subject.

As illustrated in FIG. 18A, a measurement apparatus 40 according to the first and second embodiments of the present disclosure includes, for example, a cord-like member. The measurement apparatus 40 hangs down from the measurement subject's neck by this cord-like member. The measurement subject can measure blood pressure by holding the measurement apparatus 40 hanging down from his/her neck with both hands.

As illustrated in FIG. 18B, for example, a display unit 401 for displaying a measurement result and a pulse wave measurement detection window 402 are provided on the front face of the measurement apparatus 40. Here, the display unit 401 corresponds to the display unit 400 illustrated in FIGS. 2 and 14, for example. Further, the pulse wave measurement detection window 402 corresponds to the pulse wave measurement detection window 103 illustrated in FIG. 8B, for example. As illustrated in FIG. 18B, while holding the measurement apparatus 40, for example, the measurement subject brings his/her right hand thumb into contact with the pulse wave measurement detection window 402, whereby the pulse wave-related data of the measurement subject is measured.

As illustrated in FIG. 18C, for example, two electrodes 403a and 403b are provided on the rear face of the measurement apparatus 40. Here, the electrodes 403 and 403b correspond to the electrodes 111a and 111b illustrated in FIGS. 3 and 15. As illustrated in FIG. 18C, while holding the measurement apparatus 40, the measurement subject brings his/her right hand index finger and left hand index finger into contact with the electrodes 403a and 403b, respectively, whereby the electrocardiography-related data of the measurement subject is measured. Based on the electrocardiography-related data and pulse wave-related data of the measurement subject, the measurement subject's blood pressure is calculated.

### 5.4. Other Modified Examples

In addition to the modified examples described above, the measurement apparatus according to the first and second embodiments of the present disclosure may also have the following configurations.

For example, in the above description, although the contact state between the electrocardiography measurement unit and the chest measurement site of the measurement subject was determined based on a resistance value between electrodes, the present technology is not limited to this example. Other configurations and methods can be used, as long as, for example, the presence/absence of contact between the electrocardiography measurement unit and the chest measurement site and the strength with which the electrocardiography measurement unit presses against the chest measurement site can be detected. For example, the contact state between the electrocardiography measurement unit and the chest measurement site may be determined based on the piezoelectricity generated by a piezoelectric element provided on a contact face between the electrocardiography measurement unit and the chest measurement site when the electrocardiography measurement unit is pressed against the chest measurement site.

Further, for example, although in the above description a case was described in which the pulse wave detection site is a finger, the present technology is not limited to this. The pulse wave detection site may be a site other than a finger. The pulse wave detection site may be any part as long as it is a part of the measurement subject's body. However, it is preferred that the pulse wave detection site is a site that is some distance away from the chest measurement site where electrocardiography measurement is performed. If the pulse wave detection site is a non-finger site, the pulse wave measurement unit may be configured so that it can be attached/detached to and from the chest contact measurement unit via a cable for signal transmission and reception or the like, so that a pulse wave is measured by bringing the pulse wave measurement unit into contact with the pulse wave detection site. For example, if the pulse wave detection site is an ear, and, as illustrated in FIGS. 10A and 12, the measurement apparatus is hanging from a cord-like member, the pulse wave detection site can be naturally held close to the ear by integrating the cable for signal transmission and reception with the cord-like member or arranging so that the cable for signal transmission and reception follows the cord-like member.

Further, for example, as illustrated in FIGS. 4 and 5, although in the above description a method for determining a pulse wave transit time (velocity) was described that used an electrocardiography waveform and a pulse wave, the present technology is not limited to this. For example, the electrocardiography information and pulse wave information can be managed in some other manner to the state illustrated in FIGS. 4 and 5. Since the pulse wave transit time (velocity) can be determined as long as there is information relating to the initial rise position of the electrocardiography waveform and pulse wave (times T1 and T2), for example, it is possible to manage just the initial rise position of the electrocardiography waveform and pulse wave as the electrocardiography and pulse wave information. By managing just the initial rise position of the electrocardiography waveform and pulse wave as the electrocardiography and pulse wave information, the amount of information that is stored can be reduced.

Moreover, for example, as illustrated in FIG. 3, although in the above description a case in which the electrocardiography measurement unit had two electrodes was described as an example, the present technology is not limited to this. An arbitrary number of two or more electrocardiography measurement electrodes may be provided. When the number of electrodes is more than two, a difference in potential determined by scanning in order the differences in potential between two electrodes arbitrarily selected from among all the electrodes and taking the difference in potential that was measured the most appropriately, can be employed as the electrocardiography information. By using such a method, the accuracy of the electrocardiography measurement can be further increased.

### 6. Measurement Apparatus Hardware Configuration

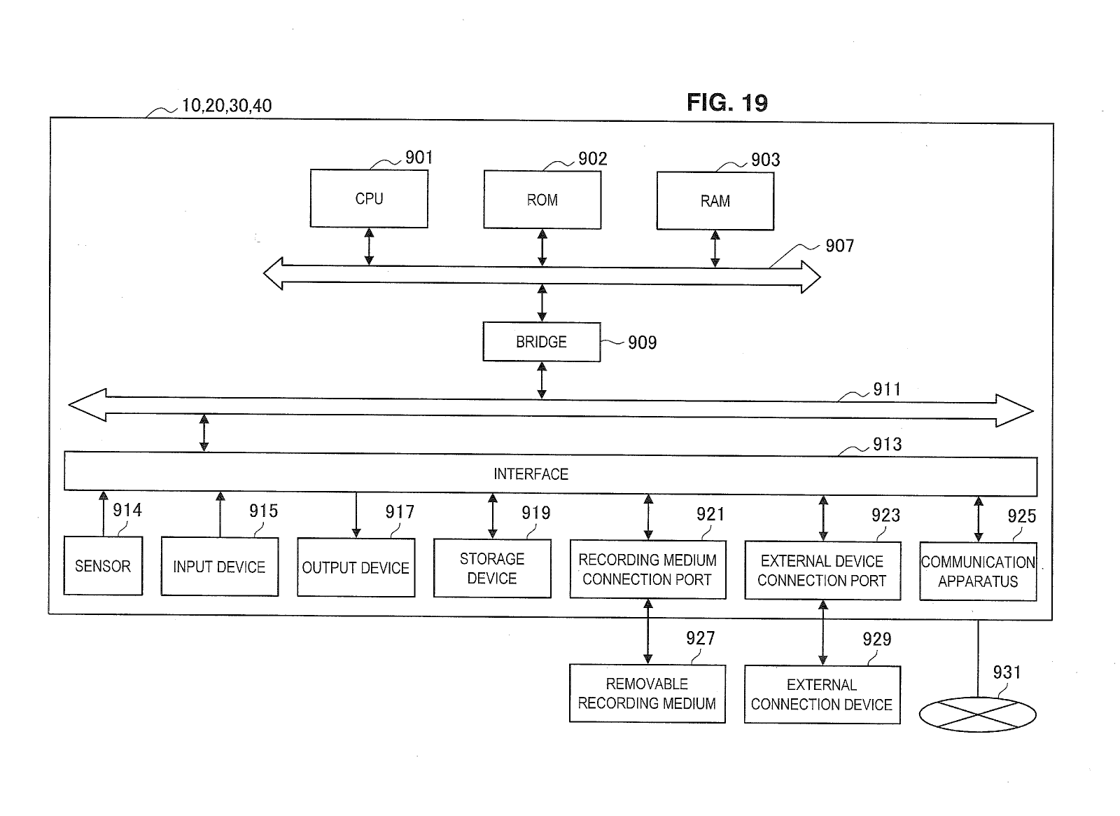

Next, the hardware configuration of the measurement apparatuses 10, 20, 30, and 40 according to the first and second embodiments of the present disclosure will be described in detail with reference to FIG. 19. FIG. 19 is a function block diagram illustrating an example of the measurement apparatuses 10, 20, 30, and 40 according to embodiments of the present disclosure.

The measurement apparatuses 10, 20, 30, and 40 include, for example, a CPU 901, a ROM 903, and a ROM 905. Further, the measurement apparatuses 10, 20, 30, and 40 further includes, for example, a host bus 907, a bridge 909, an external bus 911, an interface 913, a sensor 914, an input device 915, an output device 917, a storage device 919, a recording medium connection port 921, an external device connection port 923, and a communication apparatus 925.

The CPU 901, which functions as, for example, a calculation processing device and a control device, controls all or a part of the operations in the measurement apparatuses 10, 20, 30, and 40 based on various programs recorded in the ROM 903, RAM 905, storage device 919, or a below-described removable recording medium 927. The CPU 901 corresponds to, in the first and second embodiments of the present disclosure, the control unit 200, for example. The ROM 903 stores programs, calculation parameters and the like used by the CPU 901, for example. The RAM 905 temporarily stores the programs to be used by the CPU 901, and parameters that appropriately change during program execution, for example. The CPU 901, ROM 903, and ROM 905 are, for example, connected to each other by the host bus 907, which is configured from an internal bus such as a CPU bus.

The host bus 907 is connected to, for example, the external bus 911, which is a PCI (peripheral component interconnect/interface) bus and the like. Further, the external bus 911 is, for example, connected via the interface 913 to the sensor 914, input device 915, output device 917, storage device 919, recording medium connection port 921, external device connection port 923, and communication apparatus 925.

Here, the interface 913 may be directly connected to the host bus 907 without going through the bridge 909 or the external device 911. Namely, various interfaces, for example, the sensor 914, input device 915, output device 917, storage device 919, recording medium connection port 921, external device connection port 923, and communication apparatus 925, may be directly connected to the internal bus. If the interface 913 is directly connected to the host bus 907, for example, the measurement apparatuses 10, 20, 30, and 40 can be configured as compact built-in hardware.

The sensor 914 is a biological sensor for measuring various types data relating to the biological activity of the measurement subject. Here, the sensor 914 corresponds to, in the first and second embodiments of the present disclosure, for example, the electrocardiography measurement unit 110, the pulse wave measurement unit 120, the body surface temperature measurement unit 130, and the heart sound measurement unit 140. Further, in addition to the above-described parts, the sensor 914 may also include various measurement devices such as a barometer, a thermometer and the like.

In addition, although not illustrated in the embodiments of the present disclosure illustrated in FIGS. 2 and 14, the measurement apparatuses 10, 20, 30, and 40 may further include the input device 915 that lets the measurement subject or a user operate the measurement apparatuses 10, 20, 30, and 40. The input device 915 may be a mouse, keyboard, touch panel, button, switch, lever or the like. The input device 915 may be, for example, a remote control device (a so-called “remote control”) that utilizes infrared rays or other radio waves, or may be an external connection device 929, such as a mobile telephone or a PDA that supports the operations of the measurement apparatuses 10, 20, 30, and 40. The input device 915 includes, for example, an input control circuit that generates an input signal based on information input by the user using the above-described operation device, and outputs the generated input signal to the CPU 901. The measurement subject or the user can input various types of data into the measurement apparatuses 10, 20, 30, and 40 and issue processing operation instructions by operating this input device 915.

The output device 917 is configured from, for example, a device that can visually notify the user of acquired information. Here, the output device 917 corresponds to, in the first and second embodiments of the present disclosure, for example, the display unit 400. Examples of the output device 917 include a display device such as a CRT display device, a liquid crystal display device, a plasma display panel device, an EL display device, a lamp and the like. The output device 917 may display results obtained based on various processes performed by the measurement apparatuses 10, 20, 30, and 40 as text or an image. Further, the output device 917 may be, for example, an audio device such as a speaker, that outputs an alarm sound or the like according to the measurement from the speaker.

The storage device 919 is a device for storing data that is configured as an example of the storage unit of the measurement apparatuses 10, 20, 30, and 40. Here, the storage device 919 corresponds to, in the first and second embodiments of the present disclosure, for example, the storage unit 300. The storage device 919 is, for example, a magnetic storage unit device such as a HDD (hard disk drive), a semiconductor storage device, an optical storage device, a magneto-optical storage device and the like. This storage device 919 can store programs and various types of data executed by the CPU 901, and various types of externally-acquired data, for example.

Further, although not illustrated in the embodiments of the present disclosure illustrated in FIGS. 2 and 14, the measurement apparatuses 10, 20, 30, and 40 may further include the storage medium connection port 921, the external device connection port 923, and the communication apparatus 925. The recording medium connection port 921 is a reader/writer for a recording medium, and is installed in the measurement apparatuses 10, 20, 30, and 40 or is externally attached. The recording medium connection port 921 can read information recorded on the removable recording medium 927, and output the read information to the RAM 905. Further, the recording medium connection port 921 can also write information onto the removable recording medium 927. Here, the recording medium connection port 921 corresponds to, in the first and second embodiments of the present disclosure, for example, the connection port described in (2.1. Configuration of the measurement apparatus).

Here, the removable recording medium 927 to be connected to the recording medium connection port 921 may be, for example, a magnetic disk, an optical disc, a magneto-optical disk, a semiconductor memory and the like. More specifically, the removable recording medium 927 may be a DVD media, HD-DVD media, Blu-ray media, a CompactFlash® (CF), a flash memory, or a SD memory card (secure digital memory card) and the like. Further, the recording medium 927 may also be an IC card (integrated circuit card) on which a non-contact IC chip is mounted, an electronic device and the like.

The external device connection port 923 is a port for directly connecting an external device to the measurement apparatuses 10, 20, 30, and 40. Examples of the external device connection port 923 include, for example, a USB (universal serial bus) port, an IEEE 1394 port, a SCSI (small computer system interface) port, an RS-232C port, an optical audio terminal, a HDMI (high-definition multimedia interface) port and the like. By connecting the external connection device 929 to the external device connection port 923, the measurement apparatuses 10, 20, 30, and 40 can directly acquire various types of data from the external connection device 929 and provides various types of data to the external connection device 929.

The communication apparatus 925 is a communication interface configured from a communication device for connecting to a network 931, for example. Here, the communication device 925 corresponds to, in the first and second embodiments of the present disclosure, for example, the communication unit described in (2.1. Configuration of the measurement apparatus). Specifically, the communication apparatus 925 may be a wired or a wireless LAN (local area network), Bluetooth®, or WUSB (wireless USB) communication card, an optical communication router, an ADSL (asymmetric digital subscriber line) router, or a modem used for various types of communication. This communication apparatus 925 can transmit and receive signals and the like based on a predetermined protocol such as TCP/IP, for example, to/from the Internet or another communication device. In addition, the network 931 connected to the communication apparatus 925 is a wired or wirelessly connected network, and may be, for example, the Internet, a home LAN, infrared communication, radio wave communication, satellite communication or the like.

In the above, an example was illustrated of hardware configuration that can be executed by the measurement apparatuses 10, 20, 30, and 40 according to embodiments of the present disclosure. The above-described constituent elements may be configured using multi-purpose parts or from hardware specialized for the function of each constituent element. Therefore, the utilized hardware configuration may be appropriately modified based on the technological level at the time of implementing the embodiments of the present disclosure.

Note that there may be produced a computer program for realizing each function of the measurement apparatuses 10, 20, 30, and 40, and the measurement system 50, according to this embodiment of the present disclosure as described above, and the computer program can be implemented in a personal computer or the like. Further, there can also be provided a computer-readable recording medium having the computer program stored therein. Examples of the recording medium include a magnetic disk, an optical disc, a magneto-optical disk, and a flash memory. Further, the computer program may be distributed via a network, without using the recording medium, for example.

### 7. Summary

As described above, the measurement apparatus, measurement method, program, storage medium, and measurement system according to the first and second embodiments of the present disclosure can obtain the following advantageous effects.

First, in the measurement apparatus according to the first and second embodiments of the present disclosure, electrocardiography-related data is measured with an electrocardiography measurement unit that is in contact with the chest, and pulse wave-related measurement data is measure with a pulse wave measurement unit. Further, a blood pressure calculation unit calculates a blood pressure value of a measurement subject based on the electrocardiography information and the pulse wave information. By having such a configuration, since electrocardiography measurement is performed at a chest measurement site, the electrocardiography measurement and the blood pressure measurement can be carried out more accurately.

Further, in the measurement apparatus according to the first and second embodiments of the present disclosure, a contact information acquisition unit 240 acquires contact information, which is information relating to a contact state between an electrocardiography measurement unit and a chest measurement site. In addition, the control unit can control so that the measurement apparatus wakes up from a power standby state or enters a power standby state based on contact information, for example, the presence/absence of contact between the electrocardiography measurement unit and the chest measurement site. Therefore, while measurement is not being performed, the measurement apparatus can be kept in a power standby state, so that power consumption can be reduced.

In addition, based on at least the contact information, the measurement state determination unit determines the reliability of at least either the electrocardiography information or the pulse wave information. The measurement data relating to biological activity, such as the electrocardiography-related measurement data and the pulse wave-related measurement data, can vary due to the contact state between the electrocardiography measurement electrode and the chest measurement site and the contact state between the pulse wave detection site and the pulse wave measurement detection window. Therefore, the accuracy of the electrocardiography measurement and/or pulse wave measurement can be improved by appropriately adjusting these contact states (contact position, pressing strength etc.) according to a reliability determined based on the contact information. Therefore, the electrocardiography measurement and pulse wave measurement can be carried out more accurately, so that more accurate blood measurement can be realized.

Moreover, in the measurement apparatus according to the first and second embodiments of the present disclosure, the electrocardiography measurement unit 110 and the pulse wave measurement unit 120 may be integrally formed. Further, if the measurement subject presses the measurement apparatus against his/her chest while touching the pulse wave measurement detection window with the pulse wave detection site, he electrocardiography-related data and pulse wave-related data are simultaneously measured, and the measurement subject's blood pressure is calculated. Therefore, since the electrocardiography measurement unit is reliably pressed against the chest measurement site, the electrocardiography measurement can be performed more accurately. In addition, the measurement apparatus 10 can be carried around while hanging near to the chest measurement site of the measurement subject from a cord-like member, or while being stuck to the chest measurement site by a wet electrode for electrocardiography measurement. Consequently, the measurement subject can carry around the measurement apparatus on a daily basis while the measurement apparatus is held near his/her chest measurement site, which enables blood pressure measurement to be casually performed. Therefore, superior user friendliness is realized.

Further, especially in the measurement apparatus according to the second embodiment of the present disclosure, a pulse wave can be measured by sandwiching the pulse wave detection site between a measurement apparatus pulse wave measurement detection window and a cover. By having this configuration, since the position of the chest measurement site is fixed, pulse wave measurement can be performed more accurately.

In addition, the measurement apparatus according to the first and second embodiments of the present disclosure may be configured so that the chest contact measurement unit includes an electrocardiography measurement unit, a pulse wave measurement unit, a body surface temperature measurement unit, and a heart sound measurement unit. By utilizing measured data relating to body surface temperature and data relating to heart sound when performing calibration, a linear relationship (P=aV+b) between pulse wave velocity and a systolic pressure value can be determined more accurately. Here, the electrocardiography measurement unit, the pulse wave measurement unit, the body surface temperature measurement unit, and the heart sound measurement unit may be integrally formed.

Moreover, the measurement apparatus according to the first and second embodiments of the present disclosure can have a wearable configuration, so that for example, measurement data relating to biological activity of the measurement subject, such as electrocardiography, pulse wave, arterial oxygen saturation, body surface temperature, heart sound and the like, can be simultaneously measured with one apparatus.

It should be understood by those skilled in the art that various modifications, combinations, sub-combinations and alterations may occur depending on design requirements and other factors insofar as they are within the scope of the appended claims or the equivalents thereof.

Additionally, the present technology may also be configured as below.

(1) A measurement apparatus including:

a blood pressure calculation unit configured to calculate a blood pressure value based on electrocardiography information relating to an electrocardiogram of a measurement subject and pulse wave information relating to a pulse wave of the measurement subject; and

a chest contact measurement unit that includes an electrocardiography measurement unit that is brought into contact with a chest of the measurement subject to measure the electrocardiogram and a pulse wave measurement unit configured to measure the pulse wave from a pulse wave detection site of the measurement subject.

(2) The measurement apparatus according to (1), further including a contact information acquisition unit configured to acquire contact information that includes information relating to a contact state between the electrocardiography measurement unit and the chest.
(3) The measurement apparatus according to (2) further including a measurement control unit configured to control measurement performed by the electrocardiography measurement unit based on the contact information.
(4) The measurement apparatus according to (2) or (3), further including a measurement state determination unit configured to determine at least one of a reliability of the electrocardiography information and a reliability of the pulse wave information based on at least the contact information.
(5) The measurement apparatus according to any one of (1) to (4), wherein the pulse wave measurement unit is provided on a face opposing a contact face between the electrocardiography measurement unit and the chest.
(6) The measurement apparatus according to any one of (1) to (5),

wherein the electrocardiography measurement unit includes at least two or more electrodes, and

wherein the pulse wave measurement unit is provided at a position corresponding to between the two electrodes.

(7) The measurement apparatus according to any one of (2) to (4),

wherein the electrocardiography measurement unit includes at least two or more electrodes, and

wherein the contact information includes information relating to impedance between the at least two electrodes.

(8) The measurement apparatus according to any one of (1) to (7),

wherein the chest contact measurement unit further includes a heart sound measurement unit configured to measure a heart sound of the measurement subject, and

wherein the blood pressure calculation unit is configured to calculate a blood pressure value by further utilizing information relating to the measured heart sound.

(9) The measurement apparatus according to any one of (1) to (8),

wherein the chest contact measurement unit further includes a body surface temperature measurement unit configured to measure a body surface temperature of the measurement subject, and

wherein the blood pressure calculation unit is configured to calculate a blood pressure value by further utilizing information relating to the measured body surface temperature.

(10) The measurement apparatus according to any one of (1) to (9), wherein the pulse wave is measured by sandwiching the pulse wave detection site between a light irradiation unit and a light incident portion in the pulse wave measurement unit.
(11) The measurement apparatus according to any one of (1) to (10), wherein the electrocardiography measurement unit includes a detachable wet electrode.
(12) The measurement apparatus according to any one of (1) to (10), wherein the electrocardiography measurement unit includes at least two or more detachable dry electrodes.
(13) The measurement apparatus according to any one of (1) to (12), wherein the electrocardiography measurement unit and the pulse wave measurement unit are integrally formed.
(14) The measurement apparatus according to any one of (1) to (12), wherein the pulse wave measurement unit is detachable from the chest contact measurement unit.
(15) A measurement method including:

acquiring pulse wave information relating to a pulse wave of a measurement subject and electrocardiography information relating to an electrocardiogram of the measurement subject input from an electrocardiography measurement unit in contact with a chest of the measurement subject; and

calculating a blood pressure value based on the pulse wave information and the electrocardiography information.

(16) A program that causes a computer to realize:

a blood pressure calculation function of calculating a blood pressure value based on electrocardiography information relating to an electrocardiogram of a measurement subject and pulse wave information relating to a pulse wave of the measurement subject; and

a chest contact measurement function that includes an electrocardiography measurement unit that is brought into contact with a chest of the measurement subject to measure the electrocardiogram and a pulse wave measurement unit configured to measure the pulse wave from a pulse wave detection site of the measurement subject.

(17) A computer-readable recording medium on which is recorded a program that causes a computer to realize:

a blood pressure calculation function of calculating a blood pressure value based on electrocardiography information relating to an electrocardiogram of a measurement subject and pulse wave information relating to a pulse wave of the measurement subject; and

a chest contact measurement function that includes an electrocardiography measurement unit that is brought into contact with a chest of the measurement subject to measure the electrocardiogram and a pulse wave measurement unit configured to measure the pulse wave from a pulse wave detection site of the measurement subject.

(18) A measurement system including:

a blood pressure calculation unit configured to calculate a blood pressure value based on electrocardiography information relating to an electrocardiogram of a measurement subject and pulse wave information relating to a pulse wave of the measurement subject; and

a chest contact measurement unit that includes an electrocardiography measurement unit that is brought into contact with a chest of the measurement subject to measure the electrocardiogram and a pulse wave measurement unit configured to measure the pulse wave from a pulse wave detection site of the measurement subject.

(19) A measurement system including:

a calculation server that includes a blood pressure calculation unit configured to calculate a blood pressure value based on electrocardiography information relating to an electrocardiogram of a measurement subject and pulse wave information relating to a pulse wave of the measurement subject; and

a measurement apparatus including a chest contact measurement unit that includes an electrocardiography measurement unit that is brought into contact with a chest of the measurement subject to measure the electrocardiogram and a pulse wave measurement unit configured to measure the pulse wave from a pulse wave detection site of the measurement subject.

The present disclosure contains subject matter related to that disclosed in Japanese Priority Patent Application JP 2012-150244 filed in the Japan Patent Office on Jul. 4, 2012, the entire content of which is hereby incorporated by reference.

What is claimed is:

1. A measurement apparatus comprising:
a blood pressure calculation unit configured to calculate a blood pressure value based on electrocardiography information relating to an electrocardiogram of a measurement subject and pulse wave information relating to a pulse wave of the measurement subject; and
a chest contact measurement unit that includes an electrocardiography measurement unit that is brought into contact with a chest of the measurement subject to measure the electrocardiogram and a pulse wave measurement unit configured to measure the pulse wave from a pulse wave detection site of the measurement subject.

2. The measurement apparatus according to claim 1, further comprising a contact information acquisition unit configured to acquire contact information that includes information relating to a contact state between the electrocardiography measurement unit and the chest.

3. The measurement apparatus according to claim 2, further comprising a measurement control unit configured to control measurement performed by the electrocardiography measurement unit based on the contact information.

4. The measurement apparatus according to claim 2, further comprising a measurement state determination unit configured to determine at least one of a reliability of the electrocardiography information and a reliability of the pulse wave information based on at least the contact information.

5. The measurement apparatus according to claim 1, wherein the pulse wave measurement unit is provided on a face opposing a contact face between the electrocardiography measurement unit and the chest.

6. The measurement apparatus according to claim 5,
wherein the electrocardiography measurement unit includes at least two or more electrodes, and
wherein the pulse wave measurement unit is provided at a position corresponding to between the two electrodes.

7. The measurement apparatus according to claim 2,
wherein the electrocardiography measurement unit includes at least two or more electrodes, and
wherein the contact information includes information relating to impedance between the at least two electrodes.

8. The measurement apparatus according to claim 1,
wherein the chest contact measurement unit further comprises a heart sound measurement unit configured to measure a heart sound of the measurement subject, and
wherein the blood pressure calculation unit is configured to calculate a blood pressure value by further utilizing information relating to the measured heart sound.

9. The measurement apparatus according to claim 1,
wherein the chest contact measurement unit further comprises a body surface temperature measurement unit configured to measure a body surface temperature of the measurement subject, and
wherein the blood pressure calculation unit is configured to calculate a blood pressure value by further utilizing information relating to the measured body surface temperature.

10. The measurement apparatus according to claim 1, wherein the pulse wave is measured by sandwiching the pulse wave detection site between a light irradiation unit and a light incident portion in the pulse wave measurement unit.

11. The measurement apparatus according to claim 1, wherein the electrocardiography measurement unit includes a detachable wet electrode.

12. The measurement apparatus according to claim 1, wherein the electrocardiography measurement unit includes at least two or more detachable dry electrodes.

13. The measurement apparatus according to claim 1, wherein the electrocardiography measurement unit and the pulse wave measurement unit are integrally formed.

14. The measurement apparatus according to claim 1, wherein the pulse wave measurement unit is detachable from the chest contact measurement unit.

15. A measurement method comprising:
acquiring pulse wave information relating to a pulse wave of a measurement subject and electrocardiography information relating to an electrocardiogram of the measurement subject input from an electrocardiography measurement unit in contact with a chest of the measurement subject; and
calculating a blood pressure value based on the pulse wave information and the electrocardiography information.

16. A program that causes a computer to realize:
a blood pressure calculation function of calculating a blood pressure value based on electrocardiography information relating to an electrocardiogram of a measurement subject and pulse wave information relating to a pulse wave of the measurement subject; and
a chest contact measurement function that includes an electrocardiography measurement unit that is brought into contact with a chest of the measurement subject to measure the electrocardiogram and a pulse wave measurement unit configured to measure the pulse wave from a pulse wave detection site of the measurement subject.

17. A computer-readable recording medium on which is recorded a program that causes a computer to realize:
a blood pressure calculation function of calculating a blood pressure value based on electrocardiography information relating to an electrocardiogram of a measurement subject and pulse wave information relating to a pulse wave of the measurement subject; and
a chest contact measurement function that includes an electrocardiography measurement unit that is brought into contact with a chest of the measurement subject to measure the electrocardiogram and a pulse wave measurement unit configured to measure the pulse wave from a pulse wave detection site of the measurement subject.

18. A measurement system comprising:
a blood pressure calculation unit configured to calculate a blood pressure value based on electrocardiography information relating to an electrocardiogram of a measurement subject and pulse wave information relating to a pulse wave of the measurement subject; and
a chest contact measurement unit that includes an electrocardiography measurement unit that is brought into contact with a chest of the measurement subject to measure the electrocardiogram and a pulse wave measurement unit configured to measure the pulse wave from a pulse wave detection site of the measurement subject.

19. A measurement system comprising:
a calculation server that includes a blood pressure calculation unit configured to calculate a blood pressure value based on electrocardiography information relating to an electrocardiogram of a measurement subject and pulse wave information relating to a pulse wave of the measurement subject; and
a measurement apparatus including a chest contact measurement unit that includes an electrocardiography measurement unit that is brought into contact with a chest of the measurement subject to measure the electrocardiogram and a pulse wave measurement unit configured to measure the pulse wave from a pulse wave detection site of the measurement subject.
